(19) United States
(12) Patent Application Publication
(10) Pub. No.: US 2019/0388611 A1
(43) Pub. Date: Dec. 26, 2019

(54) INFUSION DEVICE

(71) Applicants: BAXTER INTERNATIONAL INC., Deerfield, IL (US); BAXTER HEALTHCARE SA, Glattpark (Opfikon) (CH)

(72) Inventors: Dustin Christopher Cawthon, Crystal Lake, IL (US); Ieng Kin Lao, Taipa (MO); Thomas Edward Dudar, Palatine, IL (US); Yuanpang Samuel Ding, Long Grove, IL (US); Ying-Cheng Lo, Long Grove, IL (US); Houzhi Luo, Wuxi (CN); Michael Patrick Morrissey, Algonquin, IL (US); Jeffrey Scott Packard, Woodstock, IL (US)

(21) Appl. No.: 16/013,228

(22) Filed: Jun. 20, 2018

(51) Int. Cl. A61M 5/148; A61M 5/168

(52) U.S. Cl. CPC A61M 5/1483; A61M 2205/8218; A61M 5/16831; A61M 5/16804

(57) ABSTRACT

An infusion device for dispensing fluid at a predetermined flow rate includes an elastic bladder, a pressure regulator, and a flow restrictor. The elastic bladder includes a bladder volume portion and a bladder outlet, and the elastic bladder stores fluid in the bladder volume portion and dispenses fluid through the outlet at a bladder pressure. The pressure regulator is in fluid communication with the outlet of the elastic bladder. The pressure regulator includes a fluid inlet and a fluid outlet. The fluid inlet is coupled to the bladder outlet to receive fluid from the bladder. The flow restrictor is in fluid communication with the fluid outlet. The flow restrictor and the pressure regulator cooperate to discharge fluid from the flow restrictor at a predetermined flow rate.

### BACKGROUND

The present disclosure relates to infusion devices generally and more particularly compact, ambulatory flexible bladder infusion pumps for administering a pharmaceutically active material. Flexible bladder infusion pumps may include elastomeric bladder infusion pumps and flexible bladder infusion pumps with external means for applying pressure to the bladder (e.g., platen pumps, piston pumps, etc.).

One of the embodiments of an ambulatory flexible bladder infusion pump, the elastomeric infusion pump, delivers a predetermined quantity of solution to a patient in a preselected time period at a low fluid flow rate. Known elastomeric infusion pumps include an elastic bladder for solution storage, which also acts as a pressure source for fluid movement, and an in-line flow restrictor to limit the flow rate of solution infused to the patient. In some embodiments, the desired solution flow rate is delivered at a desired and constant rate during the entire infusion therapy. However, the flow rate of current elastomeric infusion pumps may display slight variations around the desired rate and/or typically changes during the infusion therapy since the pressure generated by the elastic bladder contraction may vary. Inconsistent pressure is caused by variations in the production of the elastomeric material of the bladder and/or the intrinsic property of the elastic material of the bladder (e.g., rubber, silicone, etc.). In general, even with tight production controls, slight variations in the material, blending and/or curing of the elastomeric material will likely lead to variations in the elastic properties of the material forming the bladder. Moreover, elastic bladders inflated with the fluid to be delivered will normally generate a high pressure at the beginning and end of delivery, and a lower pressure during the middle of delivery. Other types of flexible bladder ambulatory pumps may exhibit similar variations in pressure due to the nature of the means for applying pressure on the bladder.

Pressure sources, such as elastic bladders may be characterized using sampling via an offline air and/or fluid pressure test. The test results are used to separate the bladders into groups exhibiting similar ranges of average bladder pressure (“ABP”). Each group may still contain bladders having slight variations in ABPs.

Similarly, flow restrictors (e.g., glass or metal cannula flow restrictors) are formed with slight variations in the dimensions of the flow passageway formed in the restrictor. Thus, in a similar manner flow restrictors are characterized using sampling via an off-line air flow test. The test results are used to sort the flow restrictors according to their respective air flow value into groups exhibiting similar values. An air flow value is an indicator of relative liquid flow resistance. Each sorted group of flow restrictors may still contain flow restrictors having a slight range of resistances for that group.

To assemble an overall pump that meets a target flow rate, a group of bladders are matched with the appropriate group of flow restrictors. For example, a group of bladders having a higher APB than another group may be matched with a group of flow restrictors having a higher flow resistance than another group. However, the variability of APBs within a group of bladders when combined with the variability within the matched group of restrictors in the finished devices may result in a batch of finished devices that deliver actual fluid flow rates with high variability around the mean and a mean that may not be at a specified target value. After the pump is assembled, the flow rate is tested and if the rate does not meet the release criteria the pump is scrapped. Even with the matching of the APB groups with the restrictor groups the variations within the two groups will sometimes cause the assembled pump to not meet the release criteria. Sometimes, 100% testing of each individual pump is not done. Instead, a finite number of pumps from the batch are flow tested prior to batch release. This may result in the entire batch being scrapped if the release criteria are not met.

Additionally, compact flexible bladder infusion pumps will exhibit varying pressures at the outlet of the infusion tubing resulting in varying flow rates if the height of the flexible bladder relative to the outlet (which is normally at the inlet to a patient's catheter) varies. For example, elevating the bladder relative to the outlet results in additional pressure at the outlet and if the flow restrictor is also proximate the outlet, then the flow rate may increase.

Although a variety of elastomeric bladder infusion pumps are known, there remains a need for an infusion pump that is simple and inexpensive from a manufacturing standpoint, yet is capable of delivering its contents at a substantially constant rate over the duration of the therapy and is close to the specified target value.

### SUMMARY

The present disclosure provides improved infusion devices and infusion device manufacturing methods. Aspects or embodiments of the subject matter described herein may be useful alone or in combination with one or more other aspect described herein. Without limiting the foregoing description, in a first primary embodiment, an infusion device for dispensing fluid at a predetermined flow rate is provided wherein the infusion device includes a flexible bladder and a tubing flow restrictor.

In another example embodiment, which may be combined with any other embodiments disclosed herein unless specified otherwise, the flexible bladder is an elastic bladder including a bladder volume portion and a bladder outlet. The bladder stores fluid in the bladder volume portion and dispenses fluid through the outlet at a bladder pressure.

In another example embodiment, which may be combined with any other embodiments disclosed herein unless specified otherwise, the flow restrictor is in fluid communication with the fluid outlet. Additionally, the flow restrictor is configured and arranged to restrict flow from the bladder outlet to maintain the discharged fluid at a predetermined outlet pressure and/or a desired flow rate.

In another example embodiment, which may be combined with any other embodiments disclosed herein unless specified otherwise, the flow restrictor is positioned on a patient line and in a further embodiment located distal the bladder and preferably near the connector to the infusion inlet connector to the patient.

In another example embodiment, which may be combined with any other embodiments disclosed herein unless specified otherwise, the flow restrictor includes a section of tubing having a length and an inside diameter. The length of tubing is sized based on a characteristic of the bladder, the characteristics of the fluid to be delivered, and/or the inside diameter of the tubing.

In another example embodiment, which may be combined with any other embodiments disclosed herein unless specified otherwise, the length of the tubing is sized to set the flow rate of the liquid passing therethrough and/or to provide the predetermined outlet pressure.

In one example embodiment, an infusion device for dispensing fluid at a predetermined flow rate is provided, wherein the infusion device includes an elastic bladder and a pressure regulator. The elastic bladder includes a bladder volume portion and a bladder outlet. The bladder is configured to store fluid in the bladder volume portion and dispense the fluid through the outlet at a bladder pressure. The pressure regulator is in fluid communication with the outlet of the elastic bladder. Additionally, the pressure regulator includes a fluid inlet and a fluid outlet. The fluid inlet is coupled to the bladder outlet to receive fluid from the bladder, while the pressure regulator is configured to discharge fluid from the fluid outlet at a predetermined outlet pressure.

In another example embodiment, which may be combined with any other embodiments disclosed herein unless specified otherwise, the infusion device includes a housing and tubing, the housing is sized and arranged to hold the elastic bladder and the tubing places the bladder outlet in fluid communication with the pressure regulator.

In another example embodiment, which may be combined with any other embodiments disclosed herein unless specified otherwise, the infusion device includes a housing sized and arranged to hold the elastic bladder and the pressure regulator.

In a second primary embodiment, which may be combined with any other embodiments disclosed herein unless specified otherwise, the pressure regulator includes an enclosure including a top housing, a chamber housing defining the fluid outlet, and a base housing defining the fluid inlet. The pressure regulator also includes a mechanical actuator, a valve, and a diaphragm. The mechanical actuator may be located within the top housing. The valve may be located within the chamber housing, in fluid communication with the fluid inlet and including a valve plug. The diaphragm may be located within the enclosure and seated between the top housing and the chamber housing. Additionally, the diaphragm may define a fluid sensing chamber forming a portion of a fluid path between the fluid inlet and the fluid outlet, wherein the diaphragm is in communication with the valve plug and the mechanical actuator and moveable there between to maintain the discharged fluid at the predetermined outlet pressure.

In another embodiment, which may be combined with any other embodiments discussed herein unless specified otherwise, the mechanical actuator includes a spring and a plunger. The spring causes the plunger to provide a downward force on the diaphragm that counteracts an upward force from fluid flowing through the fluid inlet.

In another embodiment, which may be combined with any other embodiments discussed herein unless specified otherwise, the spring may be adjustable to change the downward force on the diaphragm to set the pressure regulator to the predetermined outlet pressure.

In a further embodiment, which may be combined with any other embodiments discussed herein unless specified otherwise, the valve includes an o-ring adapted to form a seal between the valve plug and a valve seat of the valve.

In other example embodiments, which may be combined with any other embodiments discussed herein unless specified otherwise, the valve includes a valve seat, the valve seat shaped to assist the valve plug to form a seal with the valve seat.

In another example embodiment, which may be combined with any other embodiments discussed herein unless specified otherwise, the valve seat has a frustoconical shape.

In another example embodiment, which may be combined with any other embodiments discussed herein unless specified otherwise, the fluid path formed by the diaphragm is opened and closed via the valve plug sealing and unsealing respectively against a valve seat.

In a further embodiment, which may be combined with any other embodiments discussed herein unless specified otherwise, the diaphragm may include a central disk portion. In a further embodiment, the central disk portion may display rigidity such that flexure during normal operation is minimized.

In a further embodiment, which may be combined with any other embodiments discussed herein unless specified otherwise, the diaphragm may contain a flexible radial portion forming a rolling configuration, which may include at least one of a half wave, a full wave, a multiple half wave, or a multiple full wave configuration.

In another embodiment, which may be combined with any other embodiments discussed herein unless specified otherwise, the infusion device further includes a flow restrictor in fluid communication with the pressure regulator. The flow restrictor may be configured and arranged to restrict flow from the fluid outlet of the pressure regulator to maintain the fluid discharged from the flow restrictor at the predetermined outlet pressure and/or a desired flow rate.

In another example embodiment, which may be combined with any other embodiments discussed herein unless specified otherwise, the flow restrictor may be configured and arranged such that the restriction of the flow rate may be varied before and/or after assembly of the infusion pump.

In another example embodiment, which may be combined with any other embodiments discussed herein unless specified otherwise, the flow restrictor includes a section of tubing having a length and an inside diameter. The length of the tubing may be sized at least in part on at least one characteristic of the bladder, the characteristics of the fluid to be delivered, and the inside diameter of the tubing.

In another example embodiment, which may be combined with any other embodiments disclosed herein unless specified otherwise, the flow restrictor includes a section of tubing having a length and an inside diameter. The length of tubing is sized based in part on a pressure set point of the pressure regulator.

In another example embodiment, which may be combined with any other embodiments discussed herein unless specified otherwise, the flow restrictor includes a section of tubing having a length and an inside diameter, and the length of the tubing may be adjusted to set the flow rate of the liquid passing there through.

In another embodiment, which may be combined with any other embodiments discussed herein unless specified otherwise, the flow restrictor includes a section of tubing having a length and an inside diameter, and the length of the tubing may be sized to provide the predetermined outlet pressure and/or the desired flow rate.

In a third primary embodiment, which may be combined with any other embodiments discussed herein unless specified otherwise, an infusion device for dispensing fluid at a predetermined flow rate includes an elastic bladder, a pressure regulator, and a flow restrictor. The elastic bladder includes a bladder volume portion and a bladder outlet, and the elastic bladder stores fluid in the bladder volume portion and dispenses fluid through the outlet at a bladder pressure. The pressure regulator may be in fluid communication with the outlet of the elastic bladder. The pressure regulator includes a fluid inlet and a fluid outlet. The fluid inlet may be fluidly coupled to the bladder outlet to receive fluid from the bladder. The flow restrictor may be in fluid communication with the fluid outlet. The flow restrictor and the pressure regulator cooperate to discharge fluid from the flow restrictor at a predetermined outlet pressure and/or flow rate.

In an example embodiment, which may be combined with any other embodiments discussed herein unless specified otherwise, the infusion device further includes a flow rate adjuster in fluid communication with the flow restrictor and the pressure regulator, which cooperate to discharge fluid from the flow rate adjuster at the predetermined outlet pressure and/or a desired flow rate.

In another example embodiment, which may be combined with any other embodiments discussed herein unless specified otherwise, the flow rate adjuster defines a first flow channel in a first portion of the flow rate adjuster and a second flow channel in a second portion of the flow rate adjuster. The first portion may be configured to rotate with respect to the second portion of the flow rate adjuster to change the length of the first flow channel through which the fluid flows, thereby changing an effective length of the adjustable fluid channel.

In another example embodiment, which may be combined with any other embodiments discussed herein unless specified otherwise, the flow rate adjuster defines a first flow channel and a second flow channel, wherein the first flow channel may extend along a circular path and the second flow channel extending along a straight path, and wherein the first flow channel and second flow channel meet at their respective distal ends.

In another example embodiment, which may be combined with any other embodiments discussed herein unless specified otherwise, the flow rate adjuster may be adapted to adjust the fluid flow rate when the first flow channel is rotated with respect to the second flow channel.

In another example embodiment, which may be combined with any other embodiments discussed herein unless specified otherwise, the first flow channel has a cross-sectional area that gradually decreases along a flow direction.

In another example embodiment, which may be combined with any other embodiments discussed herein unless specified otherwise, the first flow channel has a circular cross-section having a diameter, and the diameter of the circular cross-section gradually decreases along the flow direction.

In another example embodiment, which may be combined with any other embodiments discussed herein unless specified otherwise, the first flow channel has a rectangular cross-section, which has a width and a depth. The cross-sectional area of the flow channel gradually decreases by narrowing the width, lessening the depth or a combination of both narrowing the width and lessening the depth.

In a fourth primary embodiment, which may be combined with any other embodiments discussed herein unless specified otherwise, an infusion device for dispensing fluid at a predetermined flow rate includes an elastic bladder, a pressure regulator, a flow restrictor, and a flow rate adjuster. The elastic bladder has a bladder volume and a bladder outlet. The bladder stores fluid in the bladder volume and dispenses fluid through the outlet at a bladder pressure. The pressure regulator may be in fluid communication with the outlet of the bladder, wherein the pressure regulator includes a fluid inlet and a fluid outlet. The fluid inlet may be coupled to the bladder outlet to receive fluid from the bladder. The flow restrictor may be coupled to the fluid outlet. Additionally, the flow restrictor, pressure regulator, and flow rate adjuster are configured to discharge fluid at a predetermined outlet pressure.

In a fifth primary embodiment, which may be combined with any other embodiments discussed herein unless specified otherwise, a method of manufacturing an infusion pump for fluid delivery at a target flow rate includes setting a pressure regulator to a predetermined pressure, fluidly communicating the pressure regulator with a flow restrictor to form a sub-assembly, fluidly communicating a gas source with an inlet of the sub-assembly, positioning a flow rate sensor between the gas source and the sub-assembly, flowing gas from the gas source through the sub-assembly, measuring the flow rate of the sub-assembly using the flow rate sensor, and reducing the length of the flow restrictor based on a difference between the measured flow rate and the target flow rate.

In a sixth primary embodiment, which may be combined with any other embodiments discussed herein unless specified otherwise, a method of manufacturing an infusion pump includes setting a pressure regulator to a predetermined pressure, fluidly communicating the pressure regulator with a flow restrictor to form a sub-assembly, determining a desired length of the flow restrictor based on an outlet pressure of the pressure regulator and an inside diameter of the flow restrictor, and adjusting the flow restrictor to the desired length.

In another example embodiment, which may be combined with any other embodiments discussed herein unless specified otherwise, the method includes providing the sub-assembly with a bladder of an elastomeric pump to form an infusion device for dispensing fluid at a predetermined flow rate and/or pressure.

In a seventh primary embodiment, which may be combined with any other embodiments discussed herein unless specified otherwise, a method of manufacturing an infusion pump includes fluidly communicating a flow restrictor with an elastic bladder to form a sub-assembly, measuring an outlet pressure of the sub-assembly, determining a desired length of the flow restrictor based on the outlet pressure of the sub-assembly and an inside diameter of the flow restrictor, and adjusting the flow restrictor to the desired length.

In another example embodiment, which may be combined with any other embodiments discussed herein unless specified otherwise, determining a desired length of the flow restrictor includes calculating an initial resistance of the tubing flow restrictor. Additionally, adjusting the flow restrictor to the desired length includes cutting the flow restrictor to achieve a target resistance.

In light of the embodiments set forth herein, it is accordingly an advantage of the present disclosure to reduce the variation of both nominal and instantaneous flow rates values to less than ±10% and preferably within ±5%.

It is another advantage of the present disclosure to produce finished infusion devices having more accurate and less variable flow rates.

It is another advantage of the present disclosure to provide infusion devices having continuous flow rate adjustment within a certain flow rate range for elastomeric pumps.

It is yet a further advantage of the present disclosure to provide a pump that is lower cost, lighter, and disposable, which does not require a battery, and which is beneficial to patients in home use settings.

It is yet another advantage of the present disclosure to be able to use air flow testing which is faster, more cost effective, and has less contamination risk (e.g., no need to use a liquid or to dry the part after calibration).

It is still a further advantage of the present disclosure to provide an infusion device able to provide fluid having a relatively constant pressure compared to the variable pressure exerted on the fluid within the bladder.

It is another advantage of the present disclosure to provide a device that minimizes pressure variations due to the head height differential from the bladder to the outlet connector to the connector to the patient.

Additional features and advantages of the disclosed manufacturing and calibration method and resulting infusion device are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIGS. 1A, 1B and 1C are side perspective views of an infusion device according to example embodiments of the present disclosure.

FIG. 2 is an elevation, cross-sectional view of a pressure regulator, flow restrictor, and flow rate adjuster according to an example embodiment of the present disclosure.

FIG. 3A is an exploded elevation, cross-sectional view of a pressure regulator according to an example embodiment of the present disclosure.

FIG. 3B is an elevation, cross-sectional view of a pressure regulator according to an example embodiment of the present disclosure.

FIG. 3C is an exploded elevation view of a mechanical actuator according to an example embodiment of the present disclosure.

FIG. 3D is an exploded elevation view of a mechanical actuator according to an example embodiment of the present disclosure.

FIGS. 3E and 3F are elevation, cross-sectional views of a pressure regulator according to the present disclosure.

FIGS. 4A and 4B are cross-sectional views of a rolling diaphragm according to the present disclosure.

FIGS. 4C, 4D, 4E and 4F are schematics of rolling diaphragms according to the present disclosure.

FIG. 5A is a perspective view of a flow restrictor according to an example embodiment of the present disclosure.

FIG. 5B is a top view of a flow restrictor according to an example embodiment of the present disclosure.

FIG. 6A is an elevation, cross-sectional view of a flow rate adjuster according to an example embodiment of the present disclosure.

FIG. 6B is a perspective view of a housing according to the present disclosure.

FIG. 7 is a block diagram of an example manufacturing and calibration process according to an example embodiment of the present disclosure.

FIG. 8 is a flow chart of one example process for assembling and calibrating an infusion device.

FIG. 9 is a flow chart of another example process for assembling and calibrating an infusion device.

FIG. 10 is a flow chart of an example process for calibrating an infusion device.

FIG. 11 is a flow chart of a further example process for assembling and calibrating an infusion device.

FIG. 12 is a flow chart of yet another example process for assembling and calibrating an infusion device

FIG. 13 is a flow chart of yet a further example process for assembling and calibrating an infusion device.

### DETAILED DESCRIPTION OF EXAMPLE EMBODIMENTS

As discussed above, an improved infusion apparatus and manufacturing/calibration method for the infusion apparatus are provided to reduce variation of both nominal and instantaneous flow rates values to be between ±5% and ±10%, which is close to the performance of typical electromechanical infusion pumps. The below disclosure relates to the design and manufacturing (e.g., assembly and calibration) of low cost, high flow rate accuracy, disposable intravenous medication (infusion) pumps, such as elastomeric intravenous infusion pumps and other flexible bladder infusion pumps. Additionally, the disclosure relates to flow rate adjustment features for such pumps used by end users.

The ambulatory elastomeric infusion pumps discussed herein deliver a predetermined quantity of solution to a patient over a preselected time period and at a low fluid flow rate. The elastomeric infusion pumps discussed herein may include two major components, an elastic bladder for solution storage, which also acts as a pressure source for fluid movement, and an in-line flow restrictor to limit the flow rate of solution infused to a patient. In an ideal situation, the solution flow rate is at a desired rate and is constant during the entire infusion therapy. However, variations in the qualities of the construction of the bladder may cause bladders showing similar dimension to vary in the pressure applied to a fluid within the interior of the bladder when inflated. In addition, the flow rate of current elastomeric infusion pumps may vary during the infusion therapy since pressure generated by elastic bladder contractions is not constant during deflation of the bladder. The inconsistency is caused by the intrinsic property of the elastic material of the bladder (e.g., rubber, silicone, etc.). In general, the elastic bladder generates a higher pressure at the beginning and near the end of the therapy.

To minimize the variations due to the quality of the construction of the bladder, typically, pressure sources, such as bladders are characterized by sampling from an offline pressure test and are grouped discretely according to their respective average bladder pressure (“ABP”). Each group may contain bladders still having a range of pressures exhibited by the bladder around this ABP. Similarly, flow restrictors (e.g., glass, plastic or metal cannula flow restrictors) are typically characterized by the results of sampling from an offline air flow test, such that they are sorted discretely into groups exhibiting similar air flow value according to their individual respective air flow value. An air flow value as used herein may be an indicator of relative liquid flow resistance.

Each sorted group of flow restrictors contains flow restrictors having a range of resistances. To assembly a device that meets a target flow rate, an appropriate bladder and flow restrictor are matched from their respective discrete groups. The infusion pumps disclosed herein provide an ability to change the resistance of the flow restrictor prior to or after assembly with the bladder. The infusion pumps disclosed herein also solve complications associated with the inherent variability within the chosen group of bladders compounded with the inherent variability within the chosen group of restrictors, resulting in a wide variety of fluid flow rates.

The pumps disclosed herein yield a constant flow rate pump that meets a specified target flow rate with high accuracy and low variability by simultaneously determining, in one embodiment, in a non-destructive manner, the characteristics of an individual pressure source in combination with the characteristics of the overall system (e.g., flow resistance). The pumps provide the ability to adjust the resistance during the manufacturing according to a measured characteristic of the pressure source. Additionally, the disclosed disposable elastomeric infusion pumps may have a flow rate accuracy of between ±5% to ±20%, and in a preferred embodiment between ±5% to ±10%.

### Infusion Pump with Flow Restrictor

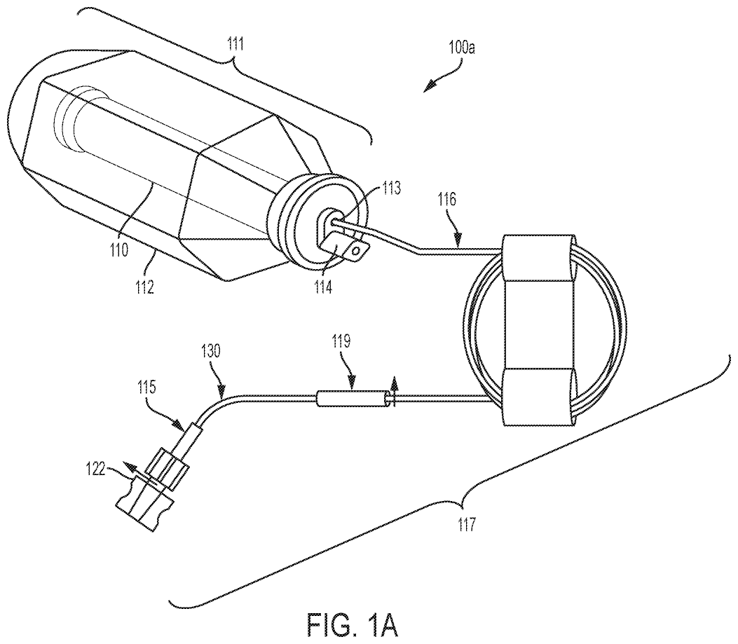

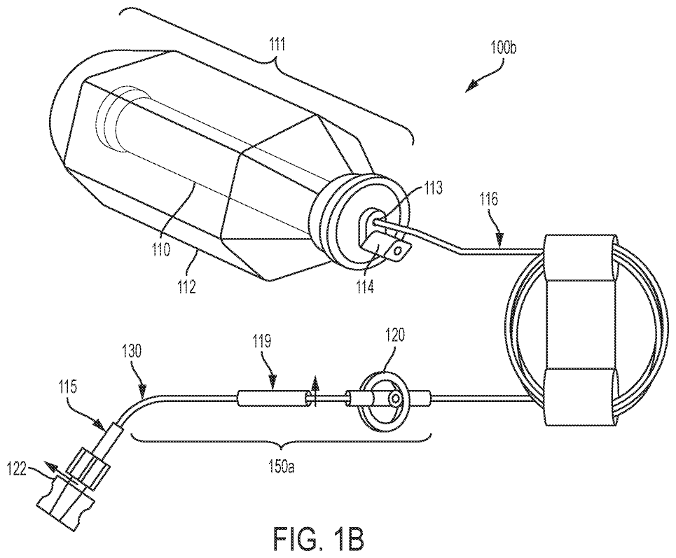

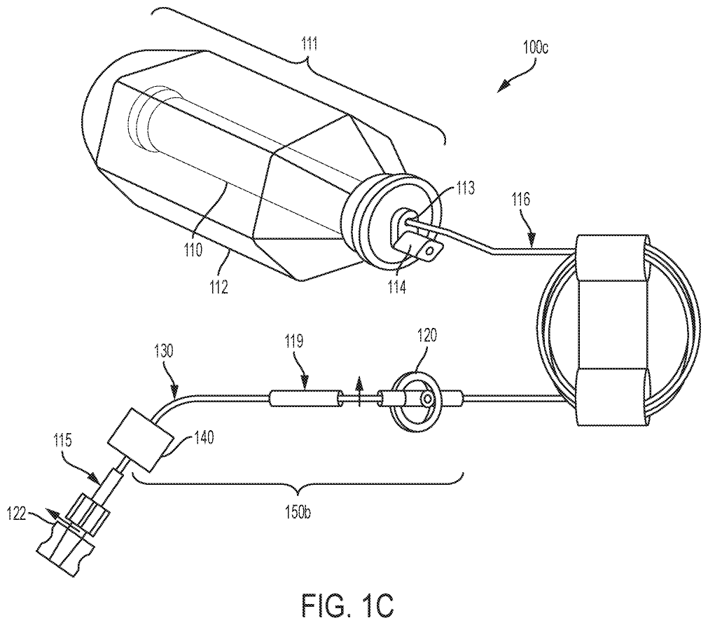

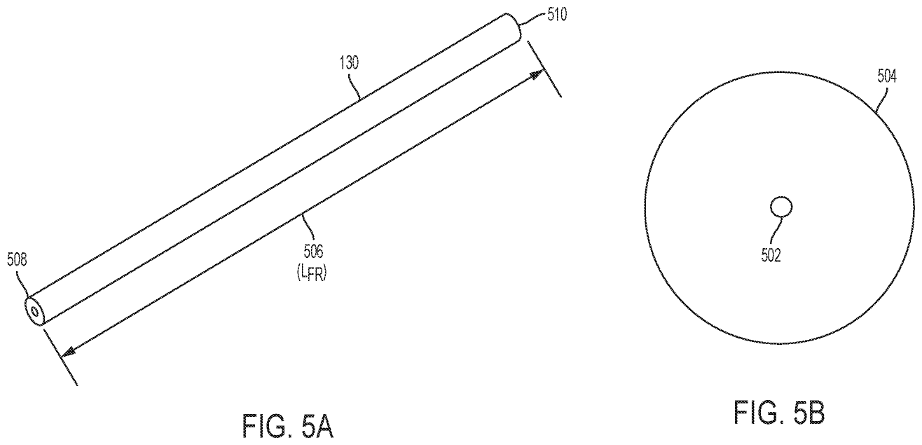

Referring to the drawings and in particular to FIGS. 1A, 1B and 1C, various embodiments of an elastomeric infusion pump are illustrated. FIG. 1A illustrates a first embodiment of an elastomeric infusion pump 100a. In the illustrated example, elastomeric infusion pump 100a includes an elastic bladder 110 and a flow restrictor 130. Elastic bladder 110 and flow restrictor 130 are in fluid communication as fluid flows from elastic bladder 110 to flow restrictor 130. The bladder 110 and housing 112 (described in more detail below) may form a sub-assembly 111. For example, fluid may flow from bladder 110 to an outlet 113 of the sub-assembly 111 and through outlet tubing 116 to flow restrictor 130. The outlet tubing 116 and flow restrictor 130 may be coupled via a connector 119. Additionally, the flow restrictor 130 may be coupled to a connector, such as a male Luer lock 115, which may include a Luer cap 122. Outlet tubing 116, connector 119, tubing flow restrictor 130, and male Luer lock 115 may form a tubing subassembly 117. In another example, the tubing subassembly may include fewer components (e.g., tubing flow restrictor 130 and male Luer lock 115). Bladder 110 may be filled with fluid (e.g., pharmaceutically active material) via fill port 114. Additional details of flow restrictor generally indicated at 130 are illustrated in FIGS. 5A and 5B and discussed in more detail below.

Optionally, infusion pump 100a may include a patient control module (“PCM”) (not shown). The PCM may allow a patient to control the delivery of fluid (e.g., medication) as described for example in U.S. Pat. No. 5,011,477 to Winchell et al. entitled, “Continuous/bolus Infusor”; U.S. Pat. No. 5,061,243 to Winchell et al. entitled, “System and Apparatus for the Patient-Controlled Delivery of a Beneficial Agent, and Set Therefor”; U.S. Pat. No. 6,027,491 to Hiejima et al. entitled, “Self-administration Device for Liquid Drugs”; and/or U.S. Pat. No. 6,936,035 to Rake et al. entitled, “Patient Controlled Drug Administration Device.

### Elastic Bladder

Infusion devices 100a, 100b and 100c include an elastomeric collapsing bladder or elastic bladder 110 disposed within a generally tubular outer casing or housing 112. The cross-sectional shape and dimension of tubular casing 112 is selected so that it limits radial outward expansion of bladder 110, thereby preventing rupture due to overfilling and overstressing bladder 110. In some embodiments, the casing 112 is rigid thereby preventing pressure applied to the exterior of the casing 112 to be transmitted to the bladder 110 thereby varying the pressure the bladder is applying to the fluid contained therein. In other embodiments, the casing 112 may be flexible but still constructed to limit the outward expansion of the bladder 110. Bladder 110 may comprise any of a variety of elastomeric compositions well known in the art, which are at least substantially inert in the presence of the pharmaceutically active material contained in the interior thereof. By inert, it may be meant that the material will not adversely react with or dissolve in the pharmaceutically active contents of filled bladder 110, nor will it catalyze or initiate a deleterious reaction of that material. Nor will deleterious chemicals migrate from the bladder into the fluid.

For example, suitable vulcanized synthetic polyisoprenes are suitable for bladder 110. Natural latex or silicone rubber having high resilience capabilities may also be used. Bladder 110 may further comprise a blend of natural and synthetic rubbers, having a high elasticity and low hysteresis. The bladder material may be selected (i) to exert sufficient force on the fluid so as to expel substantially all of the contents of the bladder after having been filled and placed in storage, typically over seven days or more, and (ii) such that the infusion pump can be stored in the assembled (stressed) but not filled state for as much as a year or longer without affecting the bladder's capability to expel its contents at a substantially constant rate.

Bladder 110 includes an elastomeric reservoir or fluid volume portion that outputs a higher pressure than the pressure set by the pressure regulator 120. The bladder pressure may depend upon any one or more of material selection, bladder wall thickness, bladder geometry, etc.

### Flow Restrictor

As illustrated in FIGS. 5A and 5B, flow restrictor 130 may be a tube, such as non-rigid or flexible plastic tubing, with an inner diameter 502 and outer diameter 504. In an example, inner diameter 502 may range from 20 microns to 1000 microns although the inner diameter 502 may vary according to the desired flow rate. Flow restrictor 130 may be sufficiently thick to prevent fluid pressure from stretching or expanding the tube. In an example, the outside diameter 504 may range from 0.09 inches (0.229 cm) to 0.10 inch (0.254 cm). The flow rate of flow restrictor 130 may be adjusted by changing the length (L_{FR}) 506 of the plastic tube. For example, the beginning length may range from 3 cm to 20 cm and can be shortened by cutting the tubing or restrictor 130 to a shorter length 506, and with the shortening, the resistance of the restrictor 130 decreases, while the flow rate increases. In an example, the final length (L_{FR}) 506 of the plastic tube may range from about 1 mm to 18 cm, although the length 506 will vary according to the inner diameter 502 and target flow rate.

In an example, flow restrictor 130 may have a constant inner diameter 502. In another example, inner diameter 502 may be variable along length 506. For example, inner diameter 502 may gradually decrease along the length 506 from a proximal end 508 to a distal end 510.

Distal end 510 and proximal end 508 of flow restrictor 130 may be configured for a connection method to any type of tube connector, such as barbed, Luer lock, threaded, compression fit, solvent or adhesive bond, etc.

Flow restrictor 130 may be made from a single material or may comprise a composite construction with, for example, at least two different materials arranged in at least two layers. The material(s) is preferably resistant to vapor transmission across its thickness. Additionally, the material(s) are preferably inert, non-toxic and biocompatible, such that the material(s) have a minimal impact on the fluid traveling though the flow restrictor 130. For example, flow restrictor 130 may be made from one or more of low density polyethylene (“LDPE”), ethylene vinyl acetate (“EVA”), and/or polyvinyl chloride (“PVC”).

### Manufacturing and Calibration of Infusion Pump with Flow Restrictor

The assembly and calibration for the above embodiment of the elastomeric infusion pump 100a provides the advantage of faster and more cost-effective construction and reduce the risk of contamination. For example, restrictor 130 does not require any type of liquid for calibration, e.g., via water. There is accordingly no need to dry parts after calibration.

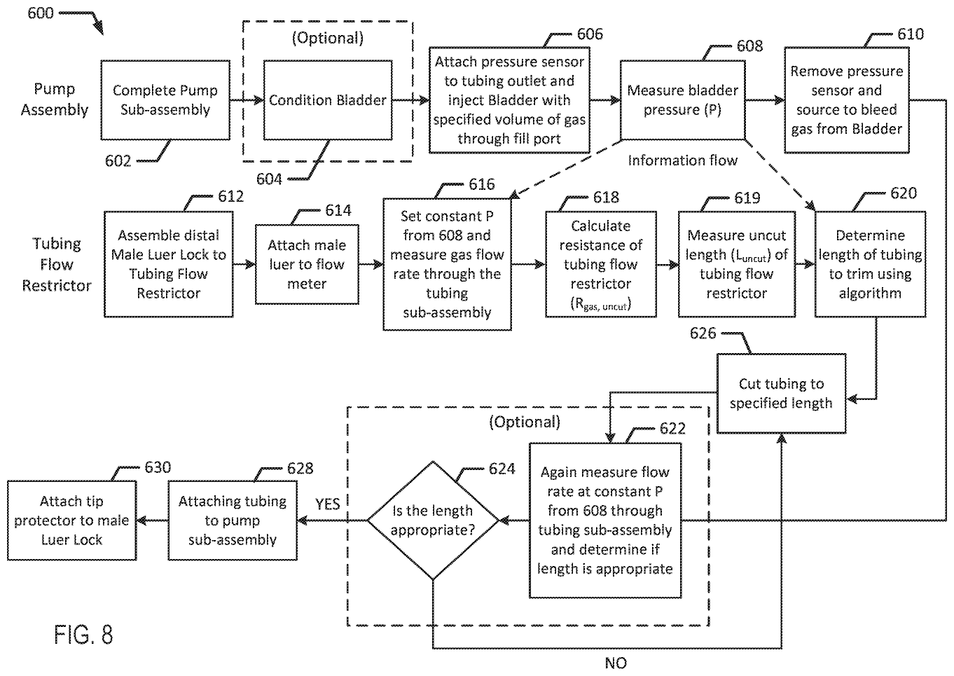

Referring now to FIG. 8, in conjunction with FIG. 1A, method 600 illustrates one embodiment for assembling an infusion device 100a with a tubing flow restrictor 130. At block 602, pump sub-assembly 111 is assembled. At block 604, bladder 110 is optionally conditioned. For example, after a bladder 110 is produced with a known normal distribution of pressure or ABP, bladder 110 may be conditioned by inflating and deflating with a gas or stretching and relaxing (e.g., via tension) a desired number of cycles. For example, bladder 110 may be conditioned by cycling bladder 110 through various gas fill and drain cycles to reduce pressure variability due to bladder hysteresis. Conditioning bladder 110 pre-stretches the bladder so that hysteresis associated with a new bladder is removed.

At block 606, a pressure sensor is connected to a tubing outlet of a sub-assembly 111, for example, to the outlet 113 of the subassembly 111 and bladder 110 is filled by injecting a specified volume of gas (e.g., with air) through its fill port 114. This specified volume of gas should be correlated to the nominal fill volume of liquid specified in the instructions for use. The volume of gas injected should result in the same bladder pressure as injecting the nominal volume of liquid. Because gas is a compressible fluid and liquid is not, the volume of injected gas may need to be larger than the volume of injected liquid to result in the same bladder pressure. This correlation can be established through experimentation, prior to manufacturing a batch of pumps. After bladder 110 is filled, bladder pressure of a pump sub-assembly 111 is measured at block 608 and that pressure is recorded. At block 610, the pressure sensor and the pressure source used to fill bladder 110 are removed to bleed the gas from bladder 110.

In parallel to assembling sub-assembly 111, the final length of tubing flow restrictor 130 may be determined by calculating an initial resistance of tubing flow restrictor 130 and then cutting the tubing to achieve the target resistance. The flow rate outputted by tubing 130 and its resistance are related based upon the Hagen-Poiseuille equation used to describe steady laminar flow of a fluid (liquid or gas) through circular tubes, where Q is the volumetric flow rate, P is the pressure drop across the tube, R is the resistance to flow across the tube. Volumetric flow rate (Q), pressure drop (P), and resistance (R) are functions of tube geometry, including (L) which is the length of the tube, (d) which is the inner diameter of the tube in combination with the viscosity (μ) of the fluid. Viscosity (μ) is a function of temperature, which may be controlled in the testing or manufacturing environment.

$$Q = \frac{P}{R} = P \frac{π {d}^{4}}{128 μ L} \qquad ( Equation 1 )$$

Based on Equation 1 and by controlling pressure (P) and the temperature of the testing environment, the flow rate varies due to viscosity of the test (i.e. calibration) fluid, such as air. Thus, a trend or correlation between the viscosity of gas, such as air and the viscosity of the medicinal liquids traveling through the tube may be determined to correct for the resulting flow rate using test fluid air. In an example, test fluids may be D5W fluids or 5% dextrose in water. Data points for the correlation may be taken when the bladder 110 is at a maximum fill, mid-point of emptying, and at the tail end of emptying. Alternatively, data points may be taken at intervals around and including the specified nominal fill volume.

For example, a look-up table may be used that correlates the flow rate of test fluid air to the flow rate of medicinal liquid D5W. There may be several different look-up tables based on the testing temperature. Alternatively, the trend may be determined prior to assembly with a test flow restrictor 130. For example, the flow rate of gas, such as air and the flow rate of liquid may be measured for the test flow restrictor 130 tube and a trend of the flow rate of air vs. the flow rate of liquid may be created by performing the same test with different pressures. To create a trend, the tests are completed at substantially the same temperature (e.g., ambient temperature, body temperature) to confirm that the fluid viscosity is constant for each data point obtained for the correlation or trend. The above measurements create the following correlation relating the flow rate of the liquid and the flow rate of gas:

Q_{liq}=fn(Q_{gas}) (Equation 2)

Note that theoretically, the ratio of gas to liquid flow rate should be inversely proportional to the ratio of gas to liquid viscosities. This can be derived from Equation 1 when P, d, and L are the same.

In an example, the conversion factor between gas test versus liquid test can be obtained experimentally. One way to determine the conversion factor between gas flow rate vs. liquid flow rate is to perform the gas/liquid test using constant pressure gas/liquid source, with the pressure at the upstream side of flow restrictor 130 controlled to be about 20% higher than the “target pressure”. Controlling the upstream pressure to a level higher than the “target pressure” ensures that the conversion factor covers the “target pressure” range. The pressure at the upstream side of flow restrictor 130 may be controlled to be more than 20% higher than the “target pressure,” for example, 30% or more.

During manufacturing, a gas such as air may be used to test the devices while a different fluid i.e. a liquid is used during therapy. Therefore, the target flow rate of a liquid during therapy will be calibrated off of a target flow rate of a gas in manufacturing. The manufacturing process targets a desired resistance with a gas using the following equation:

$${R}_{gas} = \frac{P}{{Q}_{gas}} \qquad ( Equation 3 )$$

At block 612, a male Luer lock 115 is assembled to the distal end of tubing flow restrictor 130 to produce a tubing subassembly 117. For example, an individual tubing flow restrictor 130 is randomly selected from a lot or batch of tubing flow restrictors produced at a targeted specific inner diameter and length resulting in a lot or batch with a known normal distribution of resistance. Then, at block 614, the male Luer is attached to a flow meter. Different types of flowmeters may be used, mass flow meters are advantageous because they are typically temperature and pressure independent.

At block 616, the flow rate through the tubing subassembly 117 is measured. For example, the gas flow rate (Q_{gas}) from Equation 3 above is measured through the tubing subassembly 117 with a specified pressure (P) from Equation 3 above. The specified pressure (P) is the pressure recorded in block 608 for the bladder assembly 111 to which the tubing flow assembly 117 is to be attached. At block 618, the resistance of the tubing flow restrictor 130 is calculated. For example, resistance (R_{gas, uncut}) of an uncut tubing flow restrictor 130 is calculated from Equation 3 by dividing the pressure (P) recorded at block 608 by the flow rate (Q_{gas,uncut}) obtained at block 616. Similarly, a desired resistance (R_{gas, cut}) is determined from Equation 3 using the pressure (P) recorded at block 608 and a desired flow rate (Q_{liquid}) based on the correlation of Q_{gas} to Q_{liquid} in Equation 2.

At block 619, the uncut length (L_{uncut}) of the tubing flow restrictor 130 is measured. Based on the pressure recorded at block 608, the length of tubing to trim from tubing flow restrictor 130 is determined at block 620. In an example, the length of tubing may be cut at block 626 prior to advancing to block 622. For example, to determine the desired cut length L_{cut} of tubing flow restrictor 130, the following equation may be used, where L_{uncut} is the measured initial length of tubing flow restrictor 130, R_{gas,uncut} is the initial gas resistance in the uncut tube, and R_{gas,cut} is the desired gas resistance in the cut tube.

$${L}_{cut} = {L}_{uncut} \frac{{R}_{gas , cut}}{{R}_{gas , uncut}} \qquad ( Equation 4 )$$

The tubing subassembly 117 may then be attached to the pump sub-assembly 111 at block 628.

Alternatively, at block 622, the flow rate through sub-assembly 117 may be measured to determine if the length of the tubing flow restrictor 130 is appropriate. If the result at diamond 624 is that the length is not appropriate, the tubing flow restrictor 130 may be cut again at block 626. The tubing flow restrictor may be cut and the flow rate may be measured in several iterations until the tubing flow restrictor 130 has the appropriate length.

If the result at diamond 624 is that the length is appropriate, the tubing is attached to the pump subassembly at block 628. At block 630, a tip protector is attached to the male Luer lock.

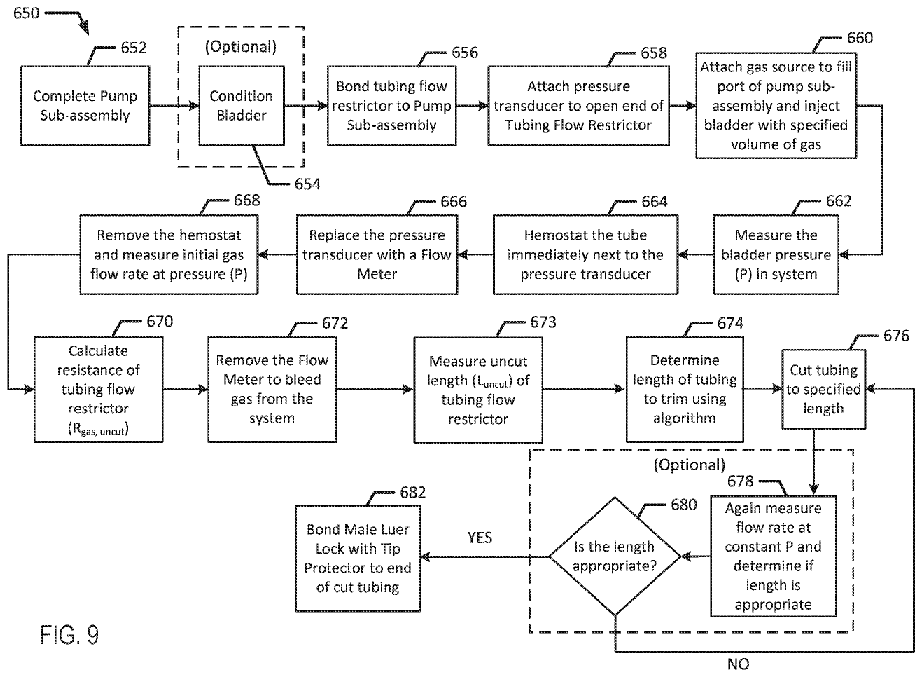

Referring now to FIG. 9, in conjunction with FIG. 1A, method 650 illustrates another embodiment for assembling an infusion device 100a with a tubing flow restrictor 130. At block 652, pump sub-assembly 111 is assembled. At block 654, bladder 110 is optionally conditioned as discussed above. For example, after a bladder 110 is randomly chosen from a lot or batch of bladders produced with a known normal distribution of pressure or ABP, bladder 110 may be conditioned by inflating and deflating with a gas or stretching and relaxing (e.g., via tension) bladder 110 a specific number of cycles. For example, bladder 110 may be conditioned by cycling the bladder through various gas fill and drain cycles to reduce bladder hysteresis. Bladder 110 may be conditioned prior to assembling the bladder into the pump assembly.

At block 656, a tubing flow restrictor 130 is bonded to sub-assembly 111 in a same manner as performed for method 600. For example, tubing flow restrictor 130 may be solvent bonded to the pump sub-assembly 111. Then, at block 658, a pressure transducer is attached to the open end of the tubing flow restrictor 130. Next, at block 660, a gas source is attached to the fill port 114 of pump sub-assembly 111 and bladder 110 is injected with a specific volume of gas. The desired volume of gas such as air may be determined in either method 600 or 650 by correlating the volume to an amount of pressure that the volume of medicinal liquid will exert on bladder 110.

After bladder 110 is filled, bladder pressure is measured at block 662. For example, pressure at the end of tubing flow restrictor 130 may be measured. The pressure (P) of the fluid is constant through the entire system since there is no flow when the measurement is made. Then, at block 664, the tubing flow restrictor is pinched to form an occlusion (e.g., by clamping a hemostat on the tube) immediately next to the pressure transducer. At block 666, the pressure transducer is replaced with a flow meter. For example, the pressure transducer may be removed and a flow meter may be attached to the open end of the tubing flow restrictor 130. In another example, the pressure transducer and flow meter may be a single instrument that provides multiple readings, and the instrument may be switched from a “pressure setting” to a “flow rate” setting. Then, at block 668, the occlusion is removed (e.g., by unclamping the hemostat) and the flow rate through the system is measured at pressure (P). For example, the occlusion is removed and the gas flow rate through the system may be measured.

Then, at block 670, the resistance of tubing flow restrictor 130 is calculated. For example, the resistance (R_{gas,uncut}) of tubing flow restrictor 130 is calculated using Equation 3 and the pressure (P) from block 662 and flow rate (Q_{gas}) obtained at block 668. At block 672, the flow meter is removed to bleed gas from the system. At block 673, the uncut length (L_{uncut}) of the tubing flow restrictor 130 is measured. Next, at block 674, the length of tubing to trim is determined. For example, the desired resistance of the system (R_{gas,cut}) may be calculated to determine the length of tubing to trim from tubing flow restrictor 130. Equation 3 may be used to determine a desirable resistance (R_{gas,cut}) with the bladder pressure (P) from block 662 and the desired flow rate (Q_{gas}) using the correlation of Q_{gas} to Q_{liquid} in Equation 2. Additionally, Equation 4 may be used to determine the desired length of flow restrictor 130. At block 676, the tubing of flow restrictor 130 is cut to the specified length.

Optionally, at block 678, the flow rate may again be measured to determine if the length of the tubing flow restrictor 130 is appropriate. If the result at diamond 680 is that the length is not appropriate, the tubing flow restrictor 130 may be cut again at block 676. The tubing flow restrictor 130 may be cut and the flow rate may be measured in several iterations until the tubing flow restrictor 130 has the appropriate length.

If the result at diamond 680 is that the length is appropriate, a male Luer with an attached tip protector is attached to the end of the cut tubing, for example by solvent bonding at block 682.

### Infusion Pump with Pressure Regulator and Flow Restrictor

Referring back to FIGS. 1B and 1C, various embodiments of an elastomeric infusion pump are illustrated. FIG. 1B illustrates a first embodiment of an elastomeric infusion pump 100b. In the illustrated example, elastomeric infusion pump 100b includes an elastic bladder 110, a pressure regulator 120, and a flow restrictor 130. Optionally, infusion pump 100b may include a patient control module (“PCM”) (not shown). The PCM may allow a patient to control a bolus delivery of fluid (e.g., medication) as described above with respect to infusion pump 100a. Pressure regulator 120 and flow restrictor 130 are in one embodiment integrated into a sub-assembly 150a. In an example, pressure regulator 120 and flow regulator 130 may be integrated or connected using a tubing connection. In another example, sub-assembly 150a may utilize a monolithic integration, where each component is formed from a single housing or structure (not shown). Elastic bladder 110, pressure regulator 120, and flow restrictor 130 are in fluid communication as fluid flows from elastic bladder 110, to pressure regulator 120, and then to flow restrictor 130. Bladder 110 may be filled with fluid (e.g., medicinal liquid or pharmaceutically active material) via fill port 114.

As illustrated in FIG. 1B, fluid may flow from bladder 110 to an outlet 113 and through outlet tubing 116 to pressure regulator 120. For example, outlet tubing 116 may place the outlet 113 (e.g., bladder outlet) in fluid communication with pressure regulator 120. The pressure regulator 120 and flow restrictor 130 may be coupled together via additional tubing and/or via connector 119.

The sub-assembly 150a of FIG. 1B including pressure regulator 120 and flow restrictor 130 may be located anywhere in between the elastic bladder 110 outlet and the patient catheter connector of the elastomeric infusion pump. Sub-assembly 150a may be installed close to (or even be integrated with) the patient catheter connector so that it may be exposed to the skin temperature of the patient to reduce the effect of temperature variation on flow rate precision. Preferably, sub-assembly 150a is installed near the distal end of the patient catheter connector close to the catheter connector-patient interface to reduce variation from the pump head height. For example, sub-assembly 150a may be taped to the patient to provide a relatively constant temperature (e.g., body temperature) during treatment. Additionally, the infusion pump 100a and 100b may be placed near the catheter-patient interface to reduce variation in the pump head. In FIG. 1C, sub-assembly 150b includes an assembly of, pressure regulator 120, flow restrictor 130 and flow rate adjustor 140.

### Pressure Regulator

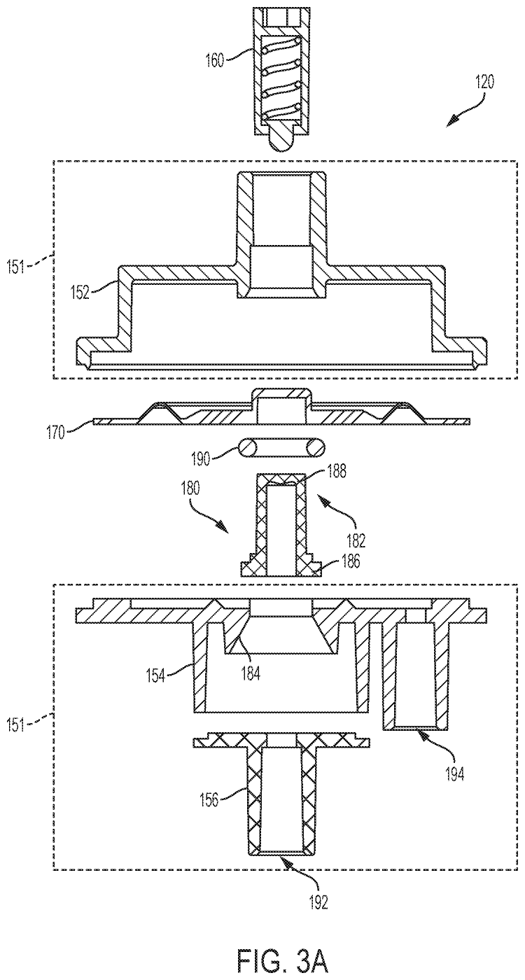

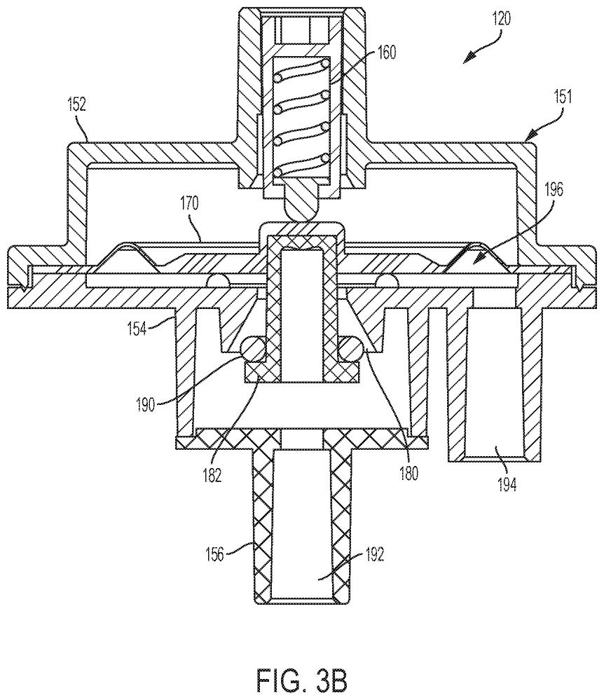

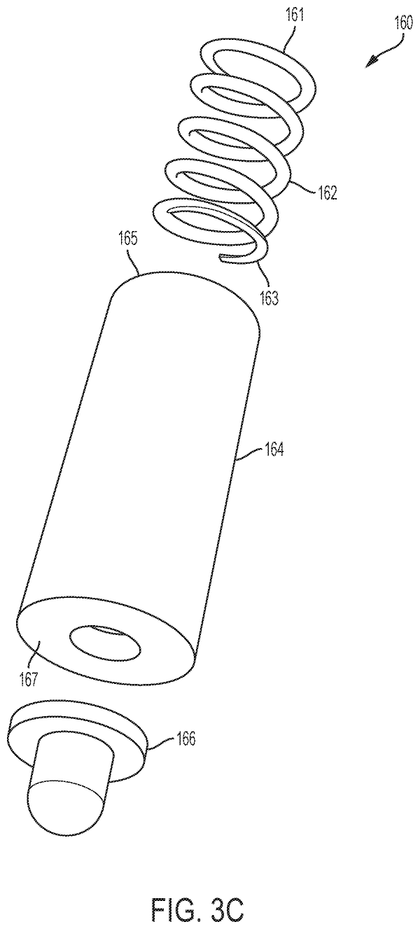

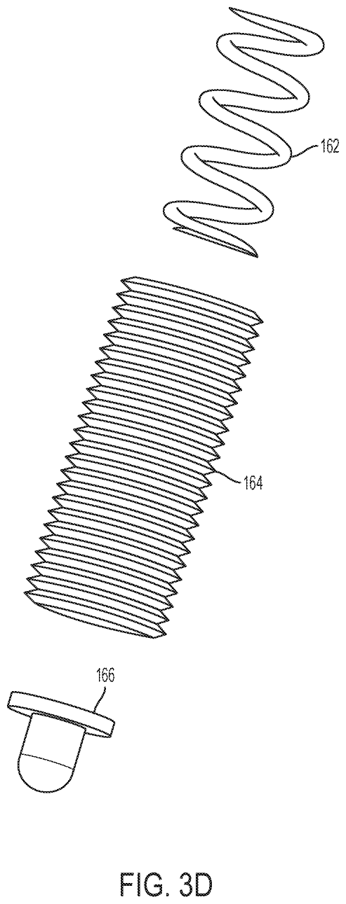

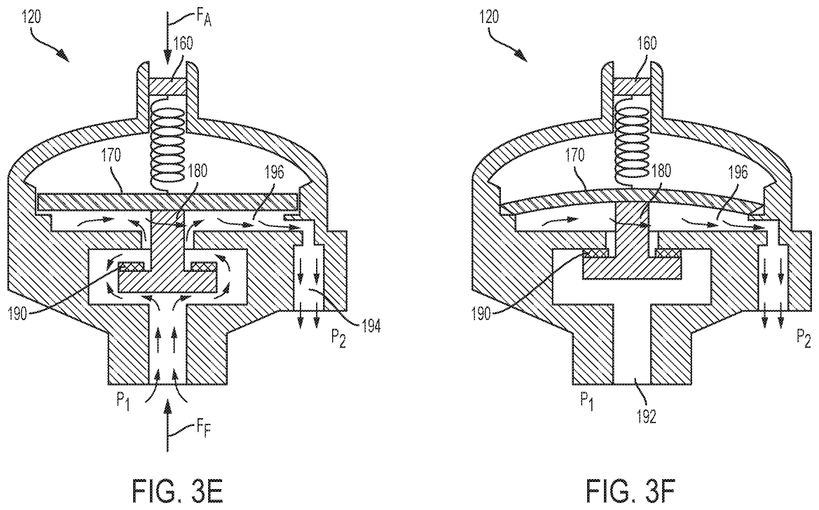

In the illustrated embodiment of FIGS. 3A and 3B, pressure regulator 120 includes an enclosure 151 having a top housing 152, a chamber housing 154, and a base housing 156. A diaphragm 170 is positioned within enclosure 151 between top housing 152 and chamber housing 154. Chamber housing 154 includes a valve seat 184 and a fluid outlet 194. A valve 180 having a valve plug 182 or piston is positioned within valve 180. Additionally, base housing 156 includes a fluid inlet 192.

Pressure regulator 120 further includes a mechanical actuator 160, such as a spring-loaded plunger (embodiments illustrated in FIGS. 3C and 3D), positioned within top housing 152. In the example illustrated in FIG. 3C, mechanical actuator 160 includes a spring 162 positioned within a plunger cylinder 164. The plunger cylinder 164 extends from a first end 165 to a second end 167 and the spring has a screw engagement end 161 and a ball engagement end 167. The screw engagement end 161 of spring 162 is in mechanical communication with a screw (not pictured) and the ball engagement end 167 of spring 162 is in mechanical communication with plunger ball 166. The screw can be rotated to extend further into plunger cylinder 164 and towards the second end 167 of plunger cylinder 164 to compress spring 162 such that a larger downward force is applied to plunger ball 166. As the screw pushes down on the screw engagement end 161 of spring 162, spring 162 compresses because the plunger ball 166 is prevented from extending beyond the second end 167 of plunger cylinder 164. Mechanical actuator 160, diaphragm 170, and valve plug 182 communicate to open and close valve 180. A temporary fluid storage chamber or sensing chamber 196 is formed between the moving diaphragm 170 and valve head 186, which provides fluid storage and a fluid path between the fluid inlet 192 and fluid outlet 194.

In the example illustrated in FIG. 3D, mechanical actuator 160 includes a spring 162 positioned within a plunger cylinder 164. The plunger cylinder 164 may be threaded and engage corresponding threads in top housing 152. For example, position of the plunger cylinder may be adjusted by rotating the plunger cylinder 164. Adjustment of the plunger cylinder may compress spring 162 such that a larger downward force is applied to plunger ball 166.

Diaphragm 170 is mechanically coupled to valve 180 and communicates with the mechanical passive actuator 160. For example, moveable diaphragm 170 acts as an element that reacts to pressure changes in fluid sensing chamber 196 and fluid inlet 192. Moveable diaphragm 170 is again mechanically coupled with valve 180 having a valve stem 188, a valve head 186, and a valve seat 184 opposite the valve head 186. Valve head 186 in the illustrated embodiment has a flat-washer shape. Moving diaphragm 170 and valve head 186 form a temporary fluid sensing chamber 196 that allows fluid to flow from fluid inlet 192 in the base housing 156 to the fluid outlet 194 in the chamber housing 154. In an example, a sealing element such as an o-ring 190 or washer may enhance the seal between valve seat 184 and valve plug 182. In an example, valve seat 184 may have a frustoconical shape to provide a stronger seal with valve plug 182. The frustoconical shape may minimize the shear forces at the interface with the valve plug. Additionally, the frustoconical shape may allow the valve plug to gradually open and close as the pressure changes. Moreover, the frustoconical shape provides a self-alignment feature between the valve and the valve seat 184.

Fluid inlet 192 and fluid outlet 194 may be located on the same side of enclosure 151 (e.g., both positioned at the bottom of the enclosure 151 as illustrated in FIG. 3A). For example, fluid inlet 192 and fluid outlet 194 may be located on the top side, the left side, the right side, etc. of the enclosure 151, so that they both extend in a same direction from regulator 120. Alternatively, fluid inlet 192 and fluid outlet 194 may be located on different sides of enclosure 151. For example, fluid inlet 192 may be positioned on the bottom of enclosure 151 while fluid outlet 194 is positioned on the top of enclosure 151. Inlet 192 and outlet 194 may be configured for any type of tube connector, such as barbed, Luer lock, threaded, compression fit, etc.

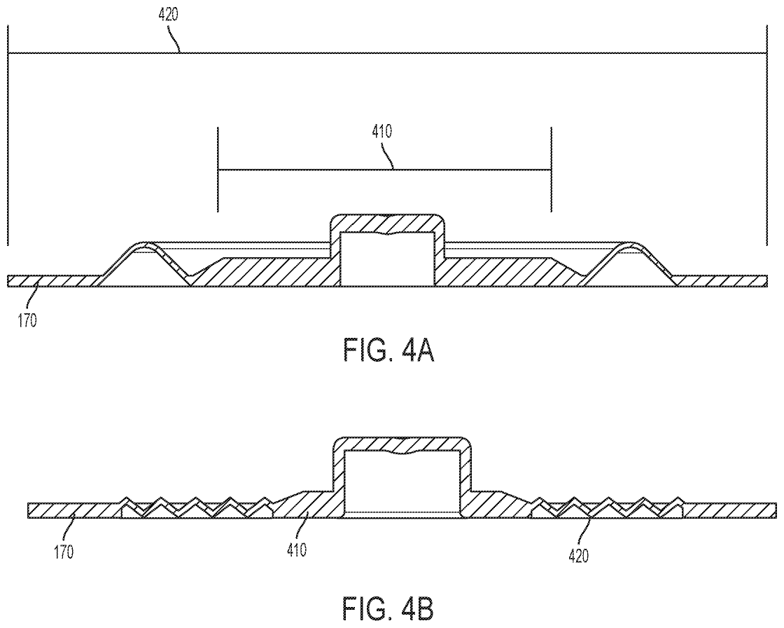

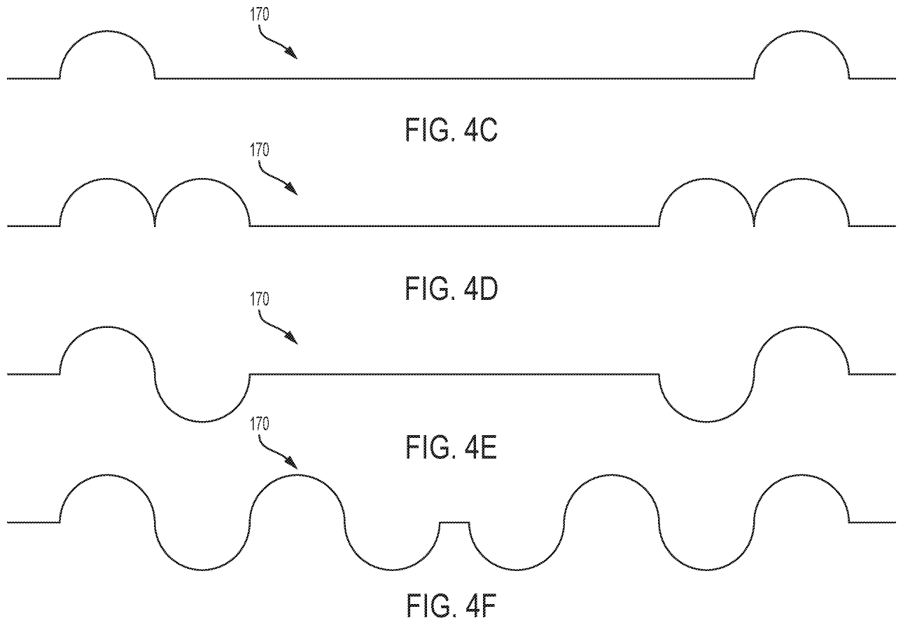

During operation, as illustrated in FIGS. 3E and 3F, fluid flows from an external upstream solution source (e.g., bladder 110) at an inlet pressure (P₁) to sensing chamber 196 located between the valve head 186 and valve seat 184. The fluid generates a vertical force to central piston region 410 (illustrated in FIG. 4A) of the moving diaphragm 170. The vertical force acting on the moving diaphragm, for example, is the sum of forces resulting from the input pressure acting on the valve 180 and pressure in the sensing chamber. Additionally, another counter balanced vertical force from the mechanical actuator 160 acts on diaphragm 170. As discussed above, the vertical force from mechanical actuator 160 may be adjusted by adjusting the height (e.g., compression) of spring 162 within mechanical actuator 160. In an example, when valve 180 is open, the force acting on the valve due to the input pressure may be zero.

As illustrated in FIGS. 3E and 3F, there are two major vertical forces acting on diaphragm 170 and valve 180, including a downward force (F_{A}) provided by spring-loaded mechanical actuator 160 and an upward force (F_{F}) due to the fluid chamber pressure (P₂) and pressure (P₁) acting on valve 180 (note that when valve 180 is open, the pressure (P₁) acting on valve 180 may be zero). The net force between each of the vertical forces (F_{A}) and (F_{F}) determines the opening and closing of valve 180.

The downward force by mechanical actuator 160 in the illustrated embodiment is determined by the spring constant of the spring in plunger 160 and/or the amount of compression of the spring. Pressure regulator 120 here may be set to a predetermined outlet pressure or “set-point” by tuning the vertical position of mechanical actuator 160 or plunger of regulator 120. Additionally, the outlet pressure may be adjusted by selecting or adjusting the spring constant of the spring in mechanical actuator 160, which may be preset by controlling the compression of the spring by adjusting the vertical position of the plunger. As illustrated, the fluid (e.g., liquid/gas) in the sensing chamber 196 produces an upward force (F_{F}) on the diaphragm 170, which is equal to the product of the chamber pressure (P₂) and the diaphragm effective area.

When force (F_{F}) equals force (F_{A}), the pressure in chamber 196 is at a pressure set-point of pressure regulator 120. This pressure set-point may be set by adjusting the vertical position of mechanical actuator 160 as discussed above.

Sensing chamber 196 of pressure regulator 120 may be initially empty and filled with atmosphere air, so that the pressure of chamber 196 is at atmospheric pressure. The upward force (F_{F}) is thus smaller than the downward force (F_{A}) (e.g., F_{F}<F_{A}), and as a result, valve 180 in pressure regulator 120 is open for fluid flow as illustrated in FIG. 3E.

When the fluid form upstream fluid source (for example, from bladder 110 of an elastomeric infusion device 100a, 100b) starts to flow into sensing chamber 196 of pressure regulator 120 via fluid inlet 192, the chamber pressure increases and the upward force (F_{F}) acting on diaphragm 170 increases. When the upstream pressure becomes larger than pressure set-point of pressure regulator 120, the upward force (F_{F}) is larger than the downward force (F_{A}) (e.g., F_{F}>F_{A}); diaphragm 170 and valve 180 move upward accordingly. As illustrated herein, diaphragm 170 may have a radial portion configured with rolling feature(s) near its peripheral edge (e.g., the “wave” feature). The rolling feature(s) near the edge rotate, while the central rigid central disk portion of diaphragm 170 translates vertically upwardly. During this transition period, valve 180 is semi-open.

Diaphragm 170 and valve 180 continue to move upwardly until valve 180 is fully closed in FIG. 3F. For example, if the fluid force (F_{F}) exceeds the force (F_{A}) produced by mechanical actuator 160, central piston region of diaphragm 170 and valve head 186 move upwardly closing valve 180, which is mechanically coupled with the central piston region of diaphragm 170. When valve 180 is fully closed, compression o-ring 190 presses against valve seat 184 of housing 154 and prevents fluid from moving from the fluid inlet 192 to fluid outlet 194. At this time, the pressure of chamber 196 is larger than the pressure set by mechanical actuator 160.

Fluid will continue to flow out from sensing chamber 196 to fluid outlet 192 due to the higher pressure in sensing chamber 196 relative to venous pressure. As the fluid flows out from sensing chamber 196 through fluid outlet 194, the pressure in sensing chamber 196 is reduced. Valve 180 remains closed as fluid flows from sensing chamber 196 through outlet 194 until the pressure in chamber 196 is reduced and reaches the pressure set-point of pressure regulator 120. At this time, the upward force (F_{F}) acting on diaphragm 170 and valve 180 equals the downward force (F_{A}).

Although the force applied by the pressure in chamber 196 equals the force of actuator 160, the pressure in chamber 196 is still higher than the downstream pressure (P₂) at outlet 194, so that fluid in fluid sensing chamber 196 flows out through fluid outlet 194. The pressure of sensing chamber 196 is reduced to a value lower than the pressure set-point of the pressure regulator, causing upward force (F_{F}) to be smaller than the downward force (F_{A}) and opening valve 180. Fluid flows from fluid inlet 192 to chamber 196 again due to the higher upstream pressure.

The sequence above is repeated for as long as the fluid pressure at the external pressure liquid source (e.g., bladder 110) is higher than the predetermined outlet pressure at fluid outlet 194. Regulator 120 causes the fluctuated pressure generated due to the contraction bladder 110 to be lessened to or close to a constant pressure in the fluid leaving outlet 194.

Moveable diaphragm 170 of pressure regulator 120 may be a rolling diaphragm having a piston structure at its center region. As illustrated in FIGS. 4A and 4B, moveable diaphragm 170 includes a central disk or piston structure 410 located within a rolling diaphragm radial ring portion 420. Central piston 410 may be formed by thickening the center region of diaphragm 170 using the same material or by co-injection molding additional elastic or non-elastomeric materials at the center region. In an example, other materials, such as non-elastomeric plastic or more rigid materials may be co-injection molded or adhered at the center region. Additionally, one or more non-elastomeric plastic component may be inserted into a flap at center region to add thickness and strength to piston structure 410. Piston structure 410 may be manufactured using any of the methods described herein. Other materials may also be utilized such as low density polyethylene, polypropylene, PVC and silicone elastomer.

The rolling diaphragm portion or ring 420 may consist of a “wave” or similar design and/or have a smaller thickness than piston structure 410. For example, moving diaphragm 170 may have a “wave” or “zig-zag” design that enables diaphragm 170 to move the piston structure 410 via un-rolling and re-rolling rather than stretching section 420. Rolling diaphragm portion or structure 420 of moving diaphragm 170 may include a “half-wave” (illustrated in FIGS. 4A and 4C), “full-wave” (illustrated in FIG. 4E), “multiple half-wave” (illustrated in FIGS. 4B and 4D), “multiple full-wave” (illustrated in FIG. 4F), or any combination of “half-wave” and “full-wave” configurations. The combination of the rolling diaphragm “wave” design and piston design advantageously reduces distortion of the center disk region or piston structure 410 as the diaphragm 170 is actuated, which allows piston structure 410 (along with valve stem 188 of valve 180) to move vertically with minimal tilting. Titling motion of the valve stem 188 may cause an incomplete fluidic seal between valve head 186 and valve seat 184, causing valve 180 to leak. In the event of extreme tilting, valve stem 188 may get stuck inside the regulator 120 causing pressure regulator 120 to malfunction.

In a preferred embodiment, moving diaphragm 170 may be a molded plastic or polymer such as low density polyethylene (PE), polyvinyl chloride (PVC), etc. O-ring 190 may be made from an elastomer, and other components of pressure regulator 120 may use medical grade moldable polymers. For example, enclosure 151 may be made from acrylonitrile butadiene styrene (ABS) plastic.

It should be appreciated that other pressure regulators may be used such as those described for example in U.S. Pat. No. 5,520,661 to Lal et al. entitled, “Fluid Flow Regulator” and U.S. Pat. No. 7,766,028 to Massengale et al. entitled, “Pressure Regulator”.

Manufacturing and Calibration of Infusion Pump with Pressure Regulator and Flow Restrictor

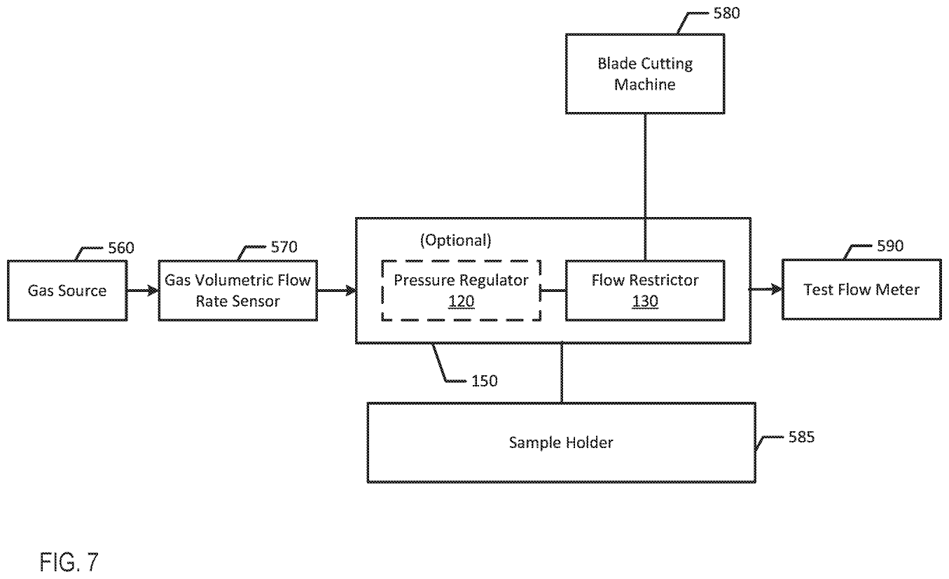

FIG. 7 illustrates a block diagram of an example arrangement to calibrate infusion devices 100a and 100b. For example, the calibration process may include a gas source 560 and a flow rate sensor 570 that are used to determine the appropriate length of flow restrictor 130. In an example, the flow restrictor 130 may optionally be connected to a pressure regulator 120 to form a sub-assembly 150a (hereinafter referred to as subassembly 150), which may be held in place by sample holder 585. The flow restrictor 130 of the subassembly 150 may then be adjusted or cut to length by blade cutting machine 580. Additionally, the calibration process may include a test flow meter 590 downstream from sub-assembly 150 to measure flow output.

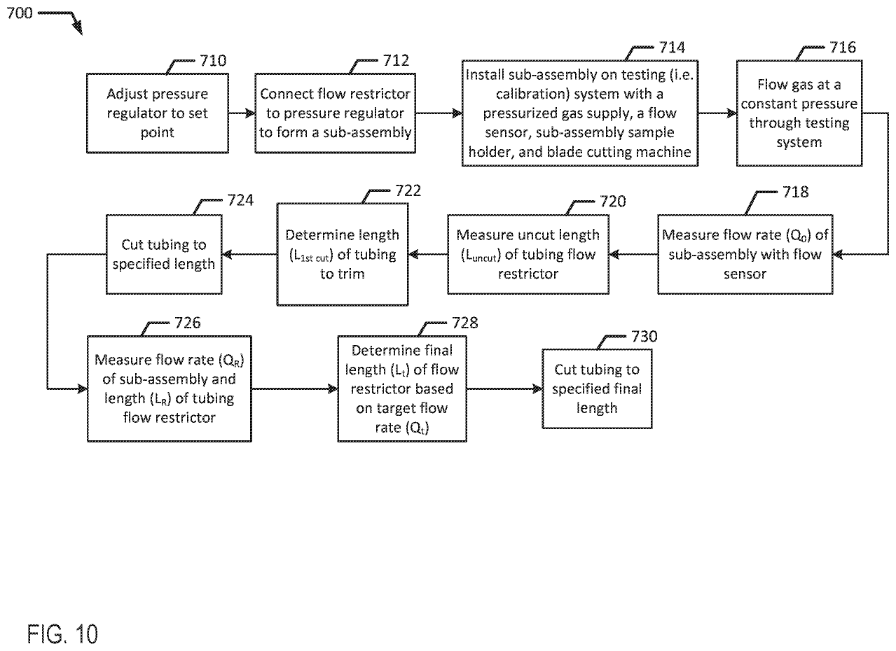

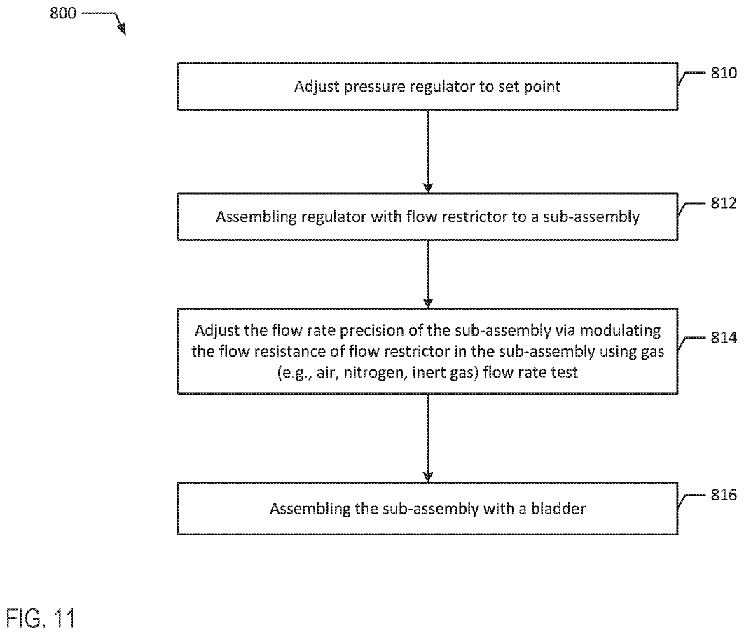

Referring now to FIG. 10 in conjunction with FIG. 1B, method 700 illustrates an embodiment for calibrating an infusion device 100b with a pressure regulator 120 and tubing flow restrictor 130. At block 710, the outlet pressure of pressure regulator 120 is roughly set to a predetermined pressure or set-point. For example, pressure regulator 120 may be set to approximately 2.5 psi (e.g., between 2.3 psi and 2.7 psi). Then, the flow restrictor 130 is connected to the outlet 194 of pressure regulator 120 forming a sub-assembly 150a.

At block 714, the sub-assembly 150a is installed on a testing system for calibration, similar to that illustrated in FIG. 7. For example, the testing system may include a pressurized gas supply (e.g., gas source 560), a flow sensor (e.g., flow sensor 570 and/or flow meter 590), a sub-assembly sample holder (e.g., sample holder 585), and a blade cutting machine (e.g., cutting machine 580). In an example, the blade cutting machine has length measurement capabilities to measure the length of flow restrictor 130.

To start the calibration, gas (e.g., air) is injected through the sub-assembly 150a at a constant pressure, for example 5 psi, such that the gas flows through all the components of the testing system at block 716. Preferably, the gas is dehumidified or kept at a constant relative humidity level. Additionally, the testing environment is preferably kept at a constant temperature, for example 23° C. during the calibration process.

At blocks 718 and 720, the initial flow rate (Q₀) of the sub-assembly 150a and the initial length or uncut length (L_{uncut}) of flow restrictor 130 are measured. In an example, the flow rate and the length may be measured at the same time. For example, the flow sensor may measure the flow rate of the sub-assembly 150a while the blade cutting machine measures the length of flow restrictor 130. Then, the length of tubing to trim (L_{1st cut}) from tubing flow restrictor 130 is determined at block 722 and the tubing is cut to a specified length or residual length (L_{R}) at block 724. The residual length (L_{R}) is the uncut length (L_{uncut}) minus the amount of tubing trimmed from the first cut (L_{1st cut}), for example (L_{R}=L_{uncut}-L_{1st cut}) The first cut length may be estimated based on a final target flow rate, the predetermined outlet pressure of pressure regulator 120 and/or previous calibrations with similar flow rates and outlet pressures. Additionally, the length of tubing to trim (L_{1st cut}) may also be estimated from Hagen-Poiseuille equation used to describe steady laminar flow of a fluid (liquid or gas) through circular tubes, which is discussed above in method 600. Preferably, the residual length (L_{R}) is longer than the final target length (L_{T}) of the flow restrictor 130. Additionally, it is preferable that the residual length (L_{R}) is 10 mm to 15 mm longer than the final target length (L_{T}).

At block 726, the residual flow rate (Q_{R}) of sub-assembly 150a and residual length (L_{R}) of tubing flow restrictor 130 are measured. As discussed above, the flow rate and length may be measured simultaneously. Since the volumetric flow rate through tubing flow restrictor 130 is inversely proportional to the tube length in a laminar flow region, the measured values of the initial flow rate (Q₀), the residual flow rate (Q_{R}), the uncut length (L_{uncut}), and the residual length (L_{R}), a correlation between the flow rate (Q) and the reciprocal of the flow restrictor length (l/L) may be determined. In an example where the inside diameter of the flow restrictor 130 is approximately constant, the correlation may be linear equation. Based on the correlation (e.g., linear equation) and the final target flow rate (Q_{T}), the final target length (L_{T}) of flow restrictor 130 may be determined at block 728. Then, at block 730, the tubing flow restrictor may be cut to the specified final length (L_{T}).

Multiple iterations of cutting and measuring residual lengths and flow rates may be performed to generate a correlation with more data points. For example, additional cuts may be made (e.g., five cuts) to create a correlation and best-fit line using each of the six data points (e.g., data point from initial measurement and 5 data points from measurements after each of the 5 cuts). Additionally, accuracy of the correlation may be further improved by precisely maintaining the temperature and relative humidity of the testing environment, eliminating or reducing any fluctuations from the pressure source, increasing precision of flow sensor and length measurement device, and reducing variation of the inner diameter of the flow restrictor 130. Even though there may be some variation of the inner diameter of the flow restrictor 130, flow rate of the sub-assembly 150a depends on the inner diameter along the entire length of flow restrictor 130, and it may be assumed that the equivalent inner diameter of the flow restrictor is approximately constant. For example, since (i) the flow rate depends on the inner diameter of the entire flow restrictor 130, (ii) the final length (L_{T}) is preferably much larger than the cut length (L_{1st cut}), and (iii) the inner diameter variation is random (i.e. not by design) along the entire flow restrictor, any effects of the diameter variation of flow restrictor 130 are likely negligent.

The assembly and calibration for the above embodiment of the elastomeric infusion pump 100b provides the advantage of faster and more cost effective construction and reduce the risk of contamination. For example, restrictor 130 and pressure regulator 120 do not require any type of liquid for calibration, e.g., via water. There is accordingly no need to dry parts after calibration.

Similar to the calibration process illustrated in FIG. 10, the manufacturing and calibration processes illustrated in FIG. 8 and FIG. 9 may also used to for an infusion device (e.g., infusion device 100b) with a pressure regulator 120 and flow restrictor 130 (some of the steps of FIGS. 8 and 9 may be redundant for infusion devices with a pressure regulator 120 and flow restrictor 130). For example, the steps of method 600 and/or method 650 may be completed after assembling tubing flow restrictor 130 and/or pressure regulator 120 to the sub-assembly 150.

For example, a way of determining the conversion factor between gas flow rate vs. liquid flow rate is to perform the gas/liquid test using constant pressure gas/liquid source, with the pressure at the upstream side of pressure regulator 120 and flow restrictor 130 sub-assembly 150 controlled to be about 20% higher than the “target pressure” selected in the pressure regulator 120.

Additionally, at block 662 of method 650, when using a pressure regulator 120 and flow restrictor 130, the pressure (P) of the fluid is constant through the entire system at this point since the fluid is traveling through pressure regulator 120 and flow restrictor 130, which equalizes the pressure (P).

Referring back to FIG. 8, at block 602, pump sub-assembly 150 may be assembled by assembling pressure regulator 120 and flow restrictor 130 together. Similarly, referring back to FIG. 9, at block 652, pump sub-assembly 150 may be assembled by assembling pressure regulator 120 and flow restrictor 130 together. For example, method 600 and/or method 650 may start by assembling pressure regulator 120 and flow restrictor 130 together, e.g., via tubing connector, compression fitting flow restrictor into fluid outlet 194 of pressure regulator 120, etc.

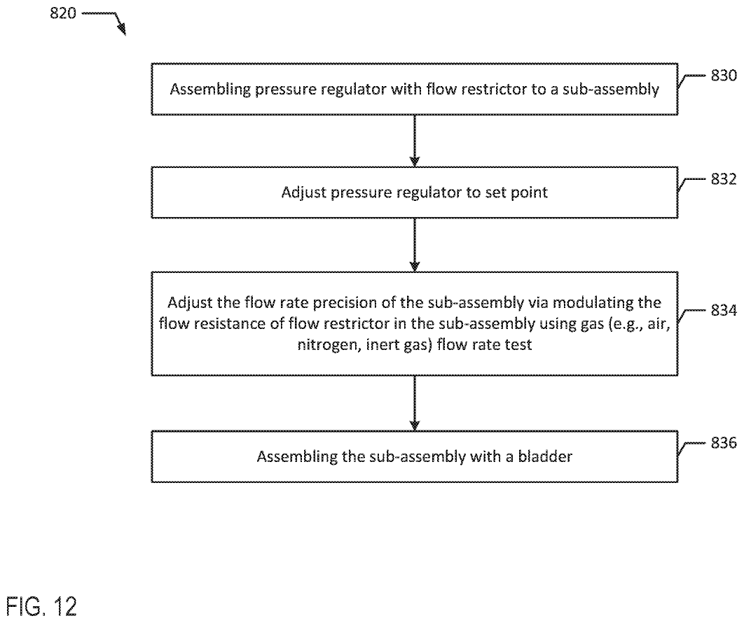

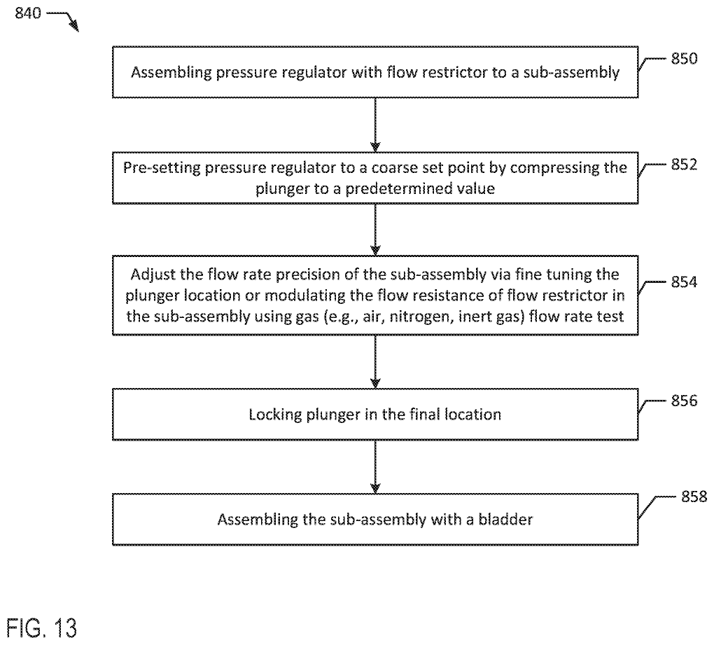

Method 800 of FIG. 11 illustrates an example sequence of an assembly and calibration process. For example, method 800 may be used in conjunction with method 700, for example method 700 may be used during the modulating steps in FIG. 11, FIG. 12 and FIG. 13. Additionally, method 800 may be used as an alternative to methods 600 or 650. First, at block 810, the pressure set-point of pressure regulator 120 is set to a target pressure, which is lower than the pressure generated by bladder contraction before the fluid in bladder 110 completely exhausts. Referring also to FIG. 3B, the force applied by the mechanical actuator 160 on the diaphragm is 170 is altered until a desired target outlet pressure is received. This can be achieved by an automatic feedback adjustment system, whereas the inlet 192 of the pressure regulator 120 is connected to a pressure source, the pressure regulator outlet 194 to a pressure sensor, and a screw plunger forming the mechanical actuator 160 of the pressure regulator to an automatic screw driver. The automatic feedback adjustment system will tighten and fine turn the vertical position of the screw plunger to the screw track (e.g., compress spring 162 within plunger cylinder 164) in the top housing 152 of the pressure regulator 120 based on the difference between the measured outlet pressure and the target outlet pressure of the pressure regulator 120 until the target outlet pressure is achieved. The outlet pressure precision of the pressure regulator using this automatic feedback adjustment system can be ranged from ±2% to ±10%.

The set-point may also be the deepest setting of the screw plunger in the screw track (e.g., maximum compression setting of spring 162 within plunger cylinder 164) in the top housing of the pressure regulator (i.e. the vertical position of the plunger; such as 5 mm). The set-point can be achieved by fixing the rotation speed and tighten time of an automatic screw driver when tightening the screw plunger to the top housing of the pressure regulator 120. The adjustment system includes an automatic screw driver without any pressure source, pressure sensor or feedback system compared to the method described above. Variation of the pressure regulator outlet pressure is relatively larger compared to the method described above due to the variation of the plunger, fluctuation of the rotation speed and tighten time etc. The outlet pressure precision of the pressure regulator using this method can be ranged from ±5% to ±20%.

The latter method to preset the pressure regulator 120 to a set-point (vertical position of the screw plunger) before assembling to a tubing flow restrictor 130 may be more adaptable for mass production with 100% inspection because it may be completed in less time and may involve an easier setup compared to the first method using the “target (outlet) pressure” as the set-point of the pressure regulator 120.

Then, at block 812, pressure regulator 120 is assembled with a flow restrictor 130 to form a sub-assembly 150. In an example, the flow rate of sub-assembly 150 is adjusted as discussed below to a specific value before final assembly with bladder 110.

For example, the sub-assembly 150 is adjusted to the flow rate set-point (e.g., within ±5%) by performing a gas (e.g., air, nitrogen, other inert gas) flow rate test at block 814. Typically, the gas flow rate setting process may only take approximately 5 to 10 seconds. After adjusting the flow rate precision of the sub-assembly, for example by adjusting the length of flow restrictor 130, the sub-assembly is assembled with the bladder at block 816. The length of flow restrictor 130 may be adjusted according to method 700, described above.

In an example in which a plastic tubing flow restrictor 130 is used (and adjuster 140 is not used), the flow rate of sub-assembly 150 may be adjusted by cutting the plastic tubing flow restrictor 130 to a desired length during the air flow test.

Method 820 of FIG. 12 illustrates an alternative sequence of an assembly and calibration process. Method 820 is similar to that described above in method 800 of FIG. 11, but the sub-assembly 150 is constructed at block 830 before adjusting the pressure regulator to a set-point at block 832. Method 820 continues with blocks 834 and 836, similar to the steps described in blocks 814 and 816 above.

Method 840 of FIG. 13 illustrates yet another example sequence of an assembly and calibration process. At block 850, pressure regulator 120 is pre-assembled with a flow restrictor 130 to form a sub-assembly 150. At block 852, the pressure regulator 120 of sub-assembly 150 is pre-set to a coarse set-point by compressing the plunger of pressure regulator 120 to a predetermined value. The final outlet pressure tolerance of sub-assembly 150 is adjusted to a desired precision by either fine tuning the plunger location of pressure regulator 120 or modulating the flow resistance of flow restrictor 130 in sub-assembly 150 using gas (e.g., air, nitrogen, inert gas) flow rate test at block 854. For example, flow resistance of flow restrictor 130 may be modulated by altering (e.g., cutting) flow restrictor 130 to a desired length. The length of flow restrictor 130 may be adjusted according to method 700, described above. At block 856, the plunger location of pressure regulator 120 is locked in place after the fine adjustment. At block 858, the sub-assembly 150 is assembled with a bladder 110.

### Infusion Pump with Pressure Regulator, Flow Restrictor, and Flow Rate Adjuster

FIG. 1C illustrates a second embodiment of an elastomeric infusion pump 100c. In FIG. 1C, elastomeric infusion pump 100c includes an elastic bladder 110, a pressure regulator 120, a flow rate adjuster 140, and a flow restrictor 130. Optionally, infusion pump 100c may include a PCM (not pictured), such as those described above. Pressure regulator 120, flow restrictor 130, and flow rate adjuster 140 are in one embodiment integrated into a sub-assembly 150b. Additionally, the flow rate adjuster 140 and flow restrictor 130 may be formed as the same integrated component or as two separate components. Elastic bladder 110, pressure regulator 120, flow rate adjuster 140, and flow restrictor 130 are in fluid communication as fluid flows from elastic bladder 110, to pressure regulator 120, to flow restrictor 130 and to flow rate adjuster 140. Flow rate adjuster 140 may be located upstream or downstream form flow restrictor 130. In an example, the flow rate adjuster 140 may have a manual flow rate control mechanism. In another example, the flow rate adjuster 140 may be battery operated and programmable.

As illustrated in FIG. 1C, fluid may flow from bladder 110 to an outlet 113 and through outlet tubing 116 to pressure regulator 120. For example, outlet tubing 116 may place the outlet 113 (e.g., bladder outlet) in fluid communication with pressure regulator 120. The pressure regulator 120 and flow restrictor 130 may be coupled together or placed in fluid communication via additional tubing and/or via connector 119. Similarly, the flow restrictor 130 and flow rate adjuster 140 may be coupled together or placed in fluid communication via additional tubing and/or connectors.

Similar to sub-assembly 150a, sub-assembly 150b is preferably installed near the distal end of the patient close to the catheter-patient interface to reduce variation from the pump head height.

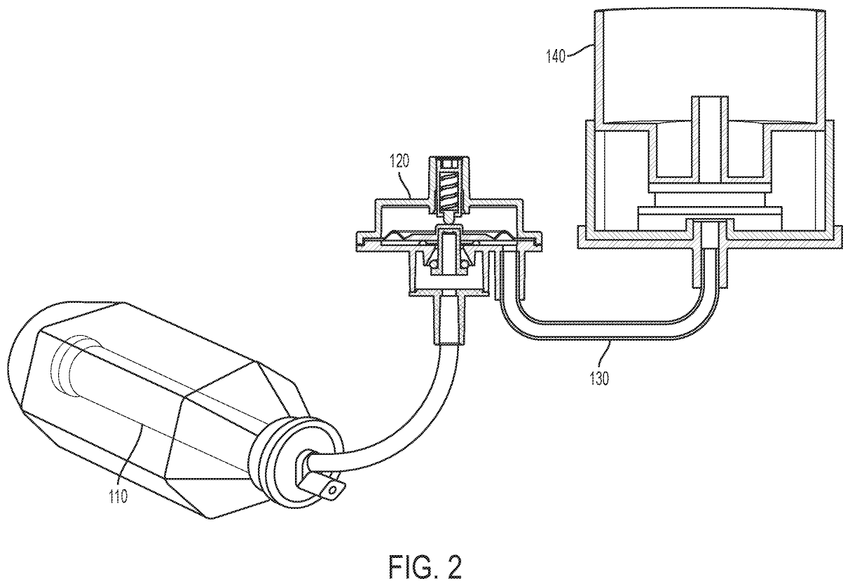

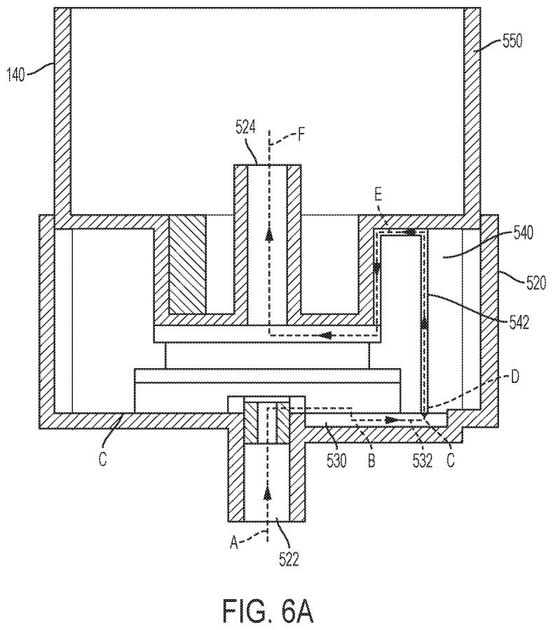

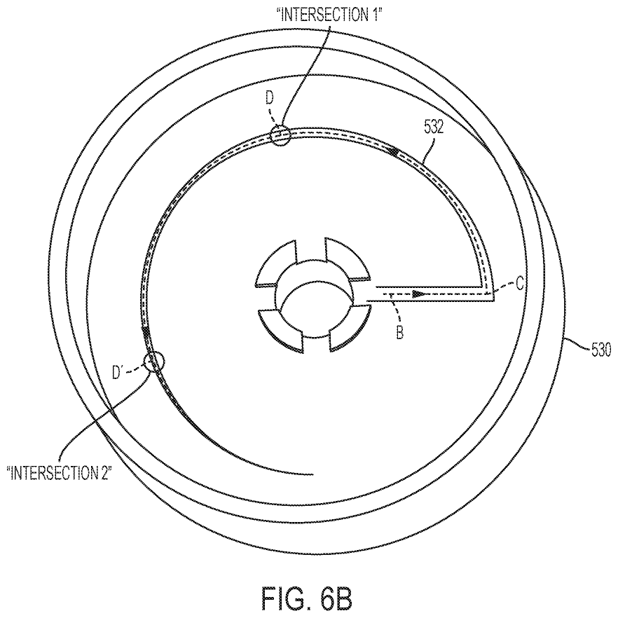

FIG. 2 illustrates sub-assembly 150b with flow rate adjuster 140. As illustrated in FIG. 2, infusion device 100c includes bladder 110 in communication with pressure regulator 120 (discussed in more detail in FIGS. 3A to 4F), which is coupled to a flow rate adjuster 140 (discussed in more detail below and in FIGS. 6A and 6B) via flow restrictor 130 (discussed in more detail in FIGS. 5A and 5B). Flow restrictor 130 may be press fit into fluid outlet 194 of pressure regulator 120. Similarly, distal end 510 of flow restrictor 130 and flow rate adjuster 140 may be configured for any type of tube connector, such as barbed, Luer lock, threaded, compression fit, etc. Additionally, pressure regulator 120, flow restrictor 130, and/or flow rate adjuster 140 may connected via solvent bonding, adhesive bonding, threaded connections, press fit connects, etc.

As illustrated in FIG. 2, the flow restrictor 130 may be located downstream of pressure regulator 120 and upstream of flow rate adjuster 140. However, flow restrictor 130 may also be located downstream of flow rate adjuster 140.

### Flow Rate Adjuster

As illustrated in FIG. 6A, one embodiment of flow rate adjuster 140 may include a bottom housing 520 defining an inlet 522 and a rotatable cap 550 defining an outlet 524. Additionally, flow rate adjuster 140 includes an interior housing 530 and a gasket holder 540. The interior housing 530 is positioned within bottom housing 520. In the illustrated embodiment, gasket holder 540 has or defines a channel 542, while interior housing 530 defines another channel 532. For example, interior housing 530 (e.g., polycarbonate housing) may be molded to have or define channel 532 and silicon gasket holder 540 may be molded to define channel 542. Interior housing 530 and gasket holder 540 may be arranged such that first channel 532 and second channel 542 intersect to allow fluid from the first channel 532 on interior housing 530 to flow along and into the second channel 542 in gasket holder 540.

Referring to FIGS. 6A and 6B, fluid may follow a flow path starting at position_A at inlet 522 to the start of channel 532 at position_B, along channel 532 on interior housing 530 from position_C to position_D or position_D′ (depending on rotation orientation), through channel 542 in gasket holder 540 to position_E, and exit through outlet 524 at position_F.

As illustrated in FIG. 6B, the diameter or cross-sectional area of channel 532 on interior housing 530 may gradually decrease along the flow direction (e.g., from position_C to position_D) as it extends counter-clockwise in FIG. 6B. For example, the diameter or cross-sectional area of channel 532 is smaller at “intersection 2” or position_D′ than it is at “intersection 1” or position_D. The length and cross-sectional area of channel 532 defined by housing 530 determines the flow resistance provided by adjusting flow rate adjuster 140. In an example, the flow resistance may be adjusted by rotating interior housing 530 in relation to silicone gasket holder 540 to adjust the amount of channel 532 that communicates with channel 542, thereby changing the effective length of the entire flow channel (e.g., channel 542 and channel 532 from position_B to position_D) and thus changing the overall resistance of the flow channel. As illustrated in FIG. 6B, the intersection of the first flow channel 532 and the second flow channel 542 may occur at “intersection 1” such that the length of the first flow channel 532 extends from position_B to position_D. Alternatively, to increase the effective length of the flow channel, the rotatable cap 550 may be rotated in a counter-clockwise direction with respect to bottom housing 520 to move the intersection from “intersection 1” to “intersection 2”. When the intersection of the first flow channel 532 and the second flow channel 542 occurs at “intersection 2”, the length of the first flow channel 532 extends from position_B to position_D′. To ensure that the first flow channel 532 and the second flow channel 542 maintain communication, the centerline of the first flow channel 532 is positioned along a circular path with a constant radius from the center of housing 530 (e.g., the center of rotation).

In an example, where flow channel 532 is circular, the diameter or radius of the circular flow channel may be reduced to reduce the cross-sectional area of channel 532. In another embodiment, flow channel 532 may have a rectangular cross-section and reducing the cross-sectional area of flow channel 532 may be accomplished by narrowing the width of flow channel 532, lessening the depth/height of flow channel 532, or a combination thereof.

Flow rate adjuster 140 allows an end user, such as pharmacists, clinicians, and patients to choose the desired flow rate such that the elastomeric pump of infusion device 100c performs similar to an electromechanical pump. The flow rate adjustment may provide a wide range of continuous flow rate adjustment, offering improved performance compared to traditional flow rate adjusters, which can typically only be adjusted to a few discrete flow rates within a narrow flow rate range, such as 0.1 to 1 ml/hr, 1 to 10 ml/hr, 10 to 100 ml/hr, 100 to 1000 ml/hr, etc. For example, the embodiments disclosed herein may allow for flow rate adjustment from 0.5 ml/hr to 100 ml/hr.

The flow rate adjuster 140 may have an indicator such as an arrow, notch, etc. on one housing while another housing has an indication of flow rates. Then, when a user rotates the housing with respect to one another, the user can visually determine what flow rate has been selected. By using flow rate adjuster 140, the accuracy of the infusion device may be improved to +1-5%.

Flow rate adjuster 140 may be used to fine-tune a flow rate from flow restrictor 130. For example, flow restrictor 130 may provide nominal flow rate accuracy and flow rate adjuster 140 may be used to fine-tune the nominal accuracy provided by flow restrictor 130. In another example, flow rate adjuster 140 may be used in place of flow restrictor 130 to adjust the flow rate of fluid exiting pressure regulator 120.

The flow rate adjustment may be manual or automatic. For example, the flow rate adjustment may be battery operated and programmable to automatically adjust the flow rate to a desired outlet flow rate or pressure via a motor. For mechanical flow rate adjustment, the flow rate may be adjusted on the flow rate adjuster itself. For example, flow rate may be adjusted by dialing (e.g., turning) interior housing 530 with respect to gasket holder 540 and or cap 550 to a desired flow rate or until the desired flow rate is achieved. Additionally, if flow rate adjuster 140 is set (or not in use), the flow rate may be adjusted on the pressure regulator 120, by changing the spring constant or vertical position of the plunger of mechanical actuator 160, and thus the outlet pressure or “set-point” of the regulator.

In an example, bottom housing 520 may be made from PVC, interior housing 530 may be made from polycarbonate, gasket holder 540 may be made from silicone, and rotatable cap 550 may be made from polycarbonate. In other examples, other materials or material combinations may be used.

It should be appreciated that the above flow rate adjustment mechanisms 130 and 140 can be applied to other elastomeric infusion pumps with different bladder configurations to extend the range or accuracy of the adjustable flow rate control of those pumps.

### Manufacturing and Calibration of Infusion Pump with Pressure Regulator, Flow Restrictor, and/or Flow Rate Adjuster

The assembly and calibration for the above embodiment of the elastomeric infusion pump 100c provides the advantage of faster and more cost effective construction and reduce the risk of contamination. For example, restrictor 130, pressure regulator 120, and flow rate adjuster 140 do not require any type of liquid for calibration, e.g., via water. There is accordingly no need to dry parts after calibration.

Further adjustment and calibration may be achieved by assembling a flow rate adjuster 140 on the infusion device. Methods 600, 650, 700, 800, 820, and/or 840 may be used to assemble an infusion device (e.g., infusion device 100c) that includes a pressure regulator 120, flow restrictor 130, and flow rate adjuster 140.

In an example in which a flow restrictor 130 with a rate adjuster 140 is used, the flow rate of the sub-assembly 150 may be adjusted by adjusting the flow channel length of the plastic flow restrictor 130 with rate adjuster 140, for example, by dialing or turning the housing 430 with respect to the gasket holder 440 to change the effective length of the flow channel.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

The invention is claimed as follows:

1. (canceled)

2. (canceled)

3. (canceled)

4. (canceled)

5. (canceled)

6. A method of manufacturing an infusion pump, the method comprising:
fluidly communicating a flow restrictor with an elastic bladder to form a sub-assembly;
measuring an outlet pressure of the sub-assembly;
determining a desired length of the flow restrictor based on the outlet pressure of the sub-assembly and an inside diameter of the flow restrictor; and
adjusting the flow restrictor to the desired length.

7. The method of claim 6, wherein determining a desired length of the flow restrictor includes calculating an initial resistance of the tubing flow restrictor, and wherein adjusting the flow restrictor to the desired length includes cutting the flow restrictor to achieve a target resistance.

8. (canceled)

9. (canceled)

10. An infusion device for dispensing fluid at a predetermined flow rate, the infusion device comprising:
an elastic bladder including a bladder volume portion and a bladder outlet, the bladder storing fluid in the bladder volume portion and dispensing fluid through the outlet at a bladder pressure; and
a pressure regulator in fluid communication with the outlet of the elastic bladder, the pressure regulator including a fluid inlet and a fluid outlet, the fluid inlet coupled to the bladder outlet to receive fluid from the bladder, the pressure regulator configured to discharge fluid from the fluid outlet at a predetermined outlet pressure.

11. The infusion device of claim 10, further comprising a housing and tubing, the housing sized and arranged to hold the elastic bladder and the tubing placing the bladder outlet in fluid communication with the pressure regulator.

12. The infusion device of claim 10, wherein the pressure regulator includes:
an enclosure including a top housing, a chamber housing defining the fluid outlet, and a base housing defining the fluid inlet;
a mechanical actuator located within the top housing;
a valve located within the chamber housing, the valve in fluid communication with the fluid inlet and including a valve plug;
a diaphragm located within the enclosure and seated between the top housing and the chamber housing, the diaphragm defining a fluid sensing chamber forming a fluid path between the fluid inlet and the fluid outlet, the diaphragm in communication with the valve plug and the mechanical actuator and moveable between the valve plug and the mechanical actuator to maintain the discharged fluid at the predetermined outlet pressure.

13. The infusion device of claim 12, wherein the mechanical actuator includes at least one of a spring and a plunger, the spring causing the plunger to provide a downward force on the diaphragm that counteracts an upward force from fluid flowing through the fluid inlet.

14. The infusion device of claim 13, wherein at least one of the spring or plunger is adjustable to change the downward force on the diaphragm to set the pressure regulator to the predetermined outlet pressure.

15. The infusion device of claim 12, wherein the valve includes an o-ring adapted to form a seal between the valve plug and a valve seat of the valve.

16. The infusion device of claim 12, wherein the valve includes a valve seat, the valve seat shaped to assist the valve plug to form a seal with the valve seat.

17. The infusion device of claim 16, wherein the valve seat has a frustoconical shape.

18. The infusion device of claim 12, wherein the fluid path formed by the diaphragm is opened and closed via the valve plug sealing and unsealing respectively against a valve seat.

19. The infusion device of claim 12, wherein the diaphragm is a rolling diaphragm.

20. The infusion device of claim 19, wherein the rolling diaphragm includes at least one of a half wave, a full wave, a multiple half wave, or a multiple full wave configuration.

21. The infusion device of claim 10, further comprising a flow restrictor in fluid communication with the pressure regulator, the flow restrictor configured and arranged to restrict flow from the fluid outlet of the pressure regulator to maintain the discharged fluid at the predetermined outlet pressure and/or a desired flow rate.

22. The infusion device of claim 21, wherein the flow restrictor includes a section of tubing having a length and an inside diameter, and wherein the length of the tubing is sized at least in part on at least one of (i) a characteristic of the bladder and (ii) a pressure set point of the pressure regulator.

23. (canceled)

24. (canceled)

25. (canceled)

26. (canceled)

27. The infusion device of claim 10, further comprising a flow rate adjuster in fluid communication with the flow restrictor, and the flow restrictor, the pressure regulator, and the flow rate adjuster cooperating to discharge fluid from the flow rate adjuster at the predetermined outlet pressure and/or a desired flow rate.

28. The infusion device of claim 27, wherein the flow rate adjuster defines a first flow channel in a first portion of the flow rate adjuster and a second flow channel in a second portion of the flow rate adjuster, wherein the first portion is configured to rotate with respect to the second portion of the flow rate adjuster to change the length of the first flow channel, thereby changing an effective length of the adjustable fluid channel.

29. The infusion device of claim 27, wherein the flow rate adjuster defines a first flow channel and a second flow channel, the first flow channel extending along a circular path and the second flow channel extending along a straight path, and wherein the first flow channel and second flow channel meet at their respective distal ends.

30. (canceled)

31. The infusion device of claim 29, wherein the first flow channel has a cross-sectional area that gradually decreases along a flow direction.

32. (canceled)

33. (canceled)

34. A method of manufacturing an infusion pump to a target flow rate, the method comprising:
setting a pressure regulator to a predetermined pressure;
fluidly communicating the pressure regulator with a flow restrictor to form a sub-assembly;
fluidly communicating a gas source with an inlet of the sub-assembly;
positioning a flow rate sensor between the gas source and the sub-assembly;
flowing gas from the gas source through the sub-assembly;
measuring the flow rate of the sub-assembly using the flow rate sensor; and
reducing the length of the flow restrictor based on a difference between the measured flow rate and the target flow rate.

35. (canceled)
